SEQUENCE LISTING (18 sequences)

SEQ ID NO: 1 - DNA, 21 bp, Artificial
  An artificially synthesized primer sequence for RT-PCR
cagaagggacagagtaaccag

SEQ ID NO: 2 - DNA, 20 bp, Artificial
  An artificially synthesized primer sequence for RT-PCR
gagatcagggttcctcactg

SEQ ID NO: 3 - DNA, 21 bp, Artificial
  An artificially synthesized primer sequence for RT-PCR
gaggtgatagcattgctttcg

SEQ ID NO: 4 - DNA, 21 bp, Artificial
  An artificially synthesized primer sequence for RT-PCR
caagtcagtgtacaggtaagc

SEQ ID NO: 5 - protein, 15 aa, Homo sapiens
IEHSVPKKQRSRKIC

SEQ ID NO: 6 - protein, 19 aa, Homo sapiens
CVKQQHQKGQSNQAQARRK

SEQ ID NO: 7 - DNA, 19 bp, Artificial
  An artificially synthesized target sequence for siRNA
gaagcagcacgacttcttc

SEQ ID NO: 8 - DNA, 19 bp, Artificial
  An artificially synthesized target sequence for siRNA
gcgcgctttgtaggattcg

SEQ ID NO: 9 - DNA, 19 bp, Artificial
  A target sequence for siRNA
ggaggagaacttctttggt

SEQ ID NO: 10 - DNA, 19 bp, Artificial
  A target sequence for siRNA
gaatctatggcaaactcaa

SEQ ID NO: 11 - DNA, 7243 bp, Homo sapiens
cctttatgcaggctcccgagcaggagatggtgcaggtgtttatccccgcccaggcagtgggcgccatcatcggcaagaaggggcagcacatcaaacagctctcccggtttgccagcgcctccatcaagattgcaccacccgaaacacctgactccaaagttcgtatggttatcatcactggaccgccagaggcccaattcaaggctcagggaagaatctatggcaaactcaaggaggagaacttctttggtcccaaggaggaagtgaagctggagacccacatacgtgtgccagcatcagcagctggccgggtcattggcaaaggtggaaaaacggtgaacgagttgcagaatttgacggcagctgaggtggtagtaccaagagaccagacccctgatgagaacgaccaggtcatcgtgaaaatcatcggacatttctatgccagtcagatggctcaacggaagatccgagacatcctggcccaggttaagcagcagcatcagaagggacagagtaaccaggcccaggcacggaggaagtgaccagcccctccctgtcccttcgagtccaggacaacaacgggcagaaatcgagagtgtgctctccccggcaggcctgagaatgagtgggaatccgggacacctgggccgggctgtagatcaggtttgcccacttgattgagaaagatgttccagtgaggaaccctgatctctcagccccaaacacccacccaattggcccaacactgtctgcccctcggggtgtcagaaattctagcgcaaggcacttttaaacgtggattgtttaaagaagctctccaggccccaccaagagggtggatcacacctcagtgggaagaaaaataaaatttccttcaggttttaaaaacatgcagagaggtgttttaatcagccttaaaggatggttcatttcttgaccttaatgtttttccaatcttcttccccctacttgggtaattgattaaaatacctccatttacggcctctttctatatttacactaatttttttatctttattgctaccagaaaaaaatgcgaacgaatgcattgctttgcttacagtattgactcaagggaaaagaactgtcagtatctgtagattaattccaatcactccctaaccaataggtacaatacggaatgaagaagaggggaaaatggggagaaagatggttaaaatacataataatccacgtttaaaaggagcgcacttgtggctgatctatgccagatcaccatcttcaaattggcacaactgaaatttccccactctgttggggcttccccaccacattcatgtccctctcccgtgtaggtttcacattatgtccaggtgcacataggtggtattgaatgctcagcagggtaggggctgaccactgtccctgattcccatcgttctcaggcggattttatatttttttaaagtctattttaatgattggatatgagcactgggaaggggacgctaactccccttgataaagtctcggttccatggaggacttgagtggccccaaaggctgccacggtgccctcaccccagcccatgtgctcccataagggctggttcctagaggcaggggttgtggggcactcccagccacggcactgttaccttggtggtgggacttggaacccaaccctgagctcccgataaagctaaagtccatcatctggcaaattcagtaaattggagagtacttgcttctgtttgtatctgagaggaatttttaactgacggcttctgtctccatgaatcattatcagcatgatgaaaggtgtgtctaaaaaacaattcagaataccagcagcattgtacagcaaggggtaaataagcttaatttattaatttaccaggcttaattaagatcccatggagtgtttagcccttgtgggagacagaagccatcagttaaatgaggttaggcctctcctcctaatatactgattgacaatgcatattagccaggtaatgcactttagctaccctggacaatgctatcaagtgtgctgggaagggaggaaggcctctctacatatggaaaagcccatgcgtggagttcccctcctttcaacattgcaacaacagtaacaacaagacaaccgcaacatgtgggcgtagtcaggcaatgctgtgtgcgaagtaaactacctcaaggtatgaagttacctcagcaattattttcctttttgttccccccaaccccattaaaaaaatttttttttgatttttgtttttttgcagcttgctgatattttatataaaaaagaaaagcaaagcaaaagagaagctgatagtcttgaatattttatttttttaatgaaaagaaaaaacaagaaagttatgtttcataatttcttacaacatgagccagtaaccctttaggaactctctatggagaacaggcctggtgggaaaggctttgggggctgcccccttaggaggaggctagtgctaagagggaaggcccaggtttgagagagcccagaggggcagagcccagagccttgtttggccctgatctctgacttctagagccccagctgctggcggctgctggaatatcctacctgataggattaaaaggcctagtggagctgggggctctcagtggttaaacaatgcccaacaaccaaccagctggcccttggtctcctctctttcctcctttggttaaagagcatctcagccagcttttcccaccagtggtgctgttgagatattttaaaatattgcctccgttttatcgaggagagaaataataactaaaaaatataccctttaaaaaaacctatatttctctgtctaaaaatatgggagctgagattccgttcgtggaaaaaagacaaggccaccctctcgccctcagagaggtccacctggtttgtcattgcaatgcttttcattttttttttttgttattgtttcatttcagttccgtcttgctattcttcctaatctatatccatagatctaaggggcaaacagatactagttaactgcccccacctctgtctccctgtcttctttagatcggtctgattgattttaaaagtggacccaaacttagggaattcttgatttagggtggctggtggcaaggaggggcaggggatatggggacgtgactgggacaggttcctgccttatcattttctccctaggacattcccttgtagcccccagaattgtctggcccaaattgaatagaagcagaaaaacatttagggataacatcaggccagtagaattaagcctctccacctgtcccaaccataaaaagggtctcccagctttccatctctggctctatatgctttatcccaaaacaaagcagataacgttcagacgtcggccatttagtaatttaaagcgaatttccagcagcaagcatgctttgatatctggttcagactatcatcaggaagaaaaaaaaatcccacagtacctgaaatgtgattgttgcagtgttcagtttccttgggggcctgctcccttcacaccttgagcccaagtccttttccgttggctgattcagctcccagaagagacgaggaagtgtgtggcaagggactggaaaacttcacttgcttggattaggcaaggctccactcattgttgatatttgcccagcaggaaaatcatgtaagttataccaccagaaagcaaaaggagcatggtttggtggttaaggtttagtgggatgaaggacctgtcttggtgggccgggccctcttgtgccccgtaggctaggtcttagggcaactccttgccctcctgctcagcacctccatttccccatccttggtgagataacaagctatcgcgaaaagcacttgggagatttggatgatttgagaagagtgacttaaaaaaaatgcttctgtgctctaagatatatatgtgtgtgtgtgtgctacatatatatttttaagaaaggaccatctctttaggatatatttttaaattctttgaaacacataaccaaaatggtttgattcactgactgactttgaagctgcatctgccagttacaccccaaatggctttaatcccctctcgggtctggttgccttttgcagtttgggttgtggactcagctcctgtgaggggtctggttaggagagagccatttttaaggacagggagttttatagcccttttctactttcctcccctcctcccagtccttatcaatcttttttcctttttcctgaccccctccttctggaggcagttgggagctatccttgtttatgcctcactattggcagaaaagaccccatttaaaacccagagaacactggagggggatgctctagttggttctgtgtccattttcctctgtgccaaagacagacagacagaggctgagagaggctgttcctgaatcaaagcaatagccagctttcgacacatacctggctgtctgaggaggaaggcctcctggaaactgggagctaagggcgaggcccttcccttcagaggctcctgggggattagggtgtggtgtttgccaagccaaggggtagggagccgagaaattggtctgtcggctcctggttgcactttggggaaggagaggaagtttggggctccaggtagctccctgttgtgggactgctctgtcccctgcccctactgcagagatagcactgccgagttcccttcaggcctggcagacgggcagtgaggaggggcctcagttagctctcaagggtgccttcccctcctcccaacccagacataccctctgccaaactgggaaccagcagtgctagtaactacctcacagagccccagagggcctgcttgagccttcttgctccacaggagaagctggtgcctctaggcaaccccttcctcccacctctcatcaggggtgggggttctcctttctttcccctgaagtgtttatggggagatcctagtggctttgccattcaaaccactcgactgtttgcctgtttcttgaaaaccagtagaagggaaacagcacagcctgtcacagtaattgcaggaagattgaagaaaaatcctcatcaatgccaggggacataaaagccatttcccttccaaatactcgacaatttagatgcagaacatttctctgtattcagacttagagtaacaccagctgaaaactgcagtttctttcctttggatacataaggcttctctatcggggtacgggacagggaggaggcctcatgtctgaagggggatttaggggcgagagccccagccctgaccctcggtcctgtgcaccgctttggggcacagtctgatggcgcctttgctggcgccttagtatggttgactccggatggacaaaagaaaaaaaattttttttcttgaatgaaatagcaggaagctcctcgggagcatgtgttttgattaaccgcaggtgatggatgctacgagtataaatggattaactacctcaatccttacagtaagattggaactaagggcagggactcatgcataagggtatgaatcccagccaggacaagtgagttgaggcttgtgccacaaaaggtttgtccttggggaacaggcaggcctgccaggatcccccccatatcgattgggctgggagggctggccatgaggtccccactttctgctttccttgcccatgtgtcacccctttggcctccagcttgtccctctctcactttctatagctttgttggaccagatggtgaggaaaggaatggcctcttcccttctagagggggctggctggagtgagacctggggcttggcctggaacccaccacacagccccaaagtcaggaagcctggggaaaccagagctgagacctcttcaacagggtttctttgagatcctacacctccattgggccctttttcagtcttcaatgggggcccagttggctctagaaggagaagaggtgaagcaggatcctttgccctgggggagtctgagggcgcggtccttggactcattcaggccgtctttgtagttgggggagttccactgggcgatcccagcccctccccacccaccctctaatggacctcctcatagaagccccatttcacttttgttttatctacctcttagcaaaacaatagataaattaggtagtggcagctccacttgcttaggttagggggggaaaaagatttctttttccaaaggaaaaaaatattaccttgagaatactttccaaaaaataaaattaaaaaaaaaaaaaccaaaaaaaaaaatttttttttaaaagggagacattttccagtgaccactggattgttttaatttcccaagcttttttttcccccataaataagtttcactctttggcgattttcttcacttgtttaagataacgtgctagctattccaacaggtaacagctttcacagtctgcccctggcctgtctcaccccatcccccaccctattcctgccagtgagtccttcctgtgcttctctcccttctcccctcccagccagctgacttcagtcacccctgtcccccctcccctgccaataagctcccccaggaataaaggctttgttttggggatgcttaaatcttgactggcacttcccggctgtgggggctggggagccacttgtaacatttctgtgcagattttatgttagccactgctatgtaaaagcacgttcaaaatgaatttcagcagattatgtgttaccataatgaataaacgtcctctatcaccatttggagtctcccttttctccaggatcttgatcctggtccccaaaaccagagtgaatcaaaagagcttcctcccctgaggcaaagtggatttgtaagcagttctgaaacatcacttactcagaagagggaacgatgtattttgatgagtgcaaattgggaagagctggaggcctactgcttgggacagttttttttttttttttttttttaaatatgagtgctagcttattctgtaattgcggcaactttgaaaattgtattttactggaaatctgccagccatcaccacccgattttgattgtatccttcctcccatcctttaatctgttcattgctttgggggaggtggggcagctggctcacacgttggagtttgttctttgatggatgaacgaacactccagttttctttcccgtgaaggttgtttcagccacaaaccacttcattttgctgtttcaatttcaaaataaaaggaaacttatattgaaagacaa

SEQ ID NO: 12 - protein, 577 aa, Homo sapiens
MNKLYIGNLNESVTPADLEKVFAEHKISYSGQFLVKSGYAFVDCPDEHWAMKAIETFSGKVELQGKRLEIEHSVPKKQRSRKIQIRNIPPQLRWEVLDSLLAQYGTVENCEQVNTESETAVVNVTYSNREQTRQAIMKLNGHQLENHALKVSYIPDEQIAQGPENGRRGGFGSRGQPRQGSPVAAGAPAKQQQVDIPLRLLVPTQYVGAIIGKEGATIRNITKQTQSKIDVHRKENAGAAEKAISVHSTPEGCSSACKMILEIMHKEAKDTKTADEVPLKILAHNNFVGRLIGKEGRNLKKVEQDTETKITISSLQDLTLYNPERTITVKGAIENCCRAEQEIMKKVREAYENDVAAMSLQSHLIPGLNLAAVGLFPASSSAVPPPPSSVTGAAPYSSFMQAPEQEMVQVFIPAQAVGAIIGKKGQHIKQLSRFASASIKIAPPETPDSKVRMVIITGPPEAQFKAQGRIYGKLKEENFFGPKEEVKLETHIRVPASAAGRVIGKGGKTVNELQNLTAAEVVVPRDQTPDENDQVIVKIIGHFYASQMAQRKIRDILAQVKQQHQKGQSNQAQARRK

SEQ ID NO: 13 - DNA, 51 bp, Artificial
  An artificially synthesized oligonucleotide for siRNA
tcccggaggagaacttctttggtttcaagagaaccaaagaagttctcctcc

SEQ ID NO: 14 - DNA, 51 bp, Artificial
  An artificially synthesized oligonucleotide for siRNA
aaaaggaggagaacttctttggttctcttgaaaccaaagaagttctcctcc

SEQ ID NO: 15 - DNA, 47 bp, Artificial
  siRNA hairpin design
ggaggagaacttctttggtttcaagagaaccaaagaagttctcctcc

SEQ ID NO: 16 - DNA, 51 bp, Artificial
  An artificially synthesized oligonucleotide for siRNA
tcccgaatctatggcaaactcaattcaagagattgagtttgccatagattc

SEQ ID NO: 17 - DNA, 51 bp, Artificial
  An artificially synthesized oligonucleotide for siRNA
aaaagaatctatggcaaactcaatctcttgaattgagtttgccatagattc

SEQ ID NO: 18 - DNA, 47 bp, Artificial
  siRNA hairpin design
gaatctatggcaaactcaattcaagagattgagtttgccatagattc

(19) United States
(12) Patent Application Publication
(10) Pub. No.: US 2011/0070245 A1
(43) Pub. Date: Mar. 24, 2011

(54) IMP-1 ONCOGENE AS A THERAPEUTIC TARGET AND PROGNOSTIC INDICATOR FOR LUNG CANCER

(75) Inventors: Yusuke Nakamura, Tokyo (DE); Yataro Daigo, Tokyo (JP); Shuichi Nakatsuru, Kanagawa (JP)

(73) Assignee: ONCOTHERAPY SCIENCE INC., KAWASAKI-SHI KANAGAWA (JP)

(21) Appl. No.: 12/377,752

(22) PCT Filed: Aug. 16, 2007
(86) PCT No.: PCT/JP2007/066324

§ 371 Date: Jul. 23, 2010

Related U.S. Application Data
(60) Provisional application No. 60/838,750, filed on Aug. 18, 2006.

(51) Int. Cl. A61K 39/395; C12Q 1/68; C12Q 1/02; G01N 33/68; A61K 31/713; A61K 31/7088; C07H 21/02; C07K 16/18; C40B 30/04; C12N 15/63; A61P 35/00

(52) U.S. Cl. 424/174.1; 435/6; 435/29; 436/501; 514/44 A; 536/24.5; 530/389.7; 506/9; 435/320.1

(57) ABSTRACT

IMP-1 was abundantly expressed in the majority of lung-cancers examined. Positive immunostaining of IMP-1 was correlated with tumor size (pT-classification; P=0.0003), non-adenocarcinoma histology (P<0.0001), low-histological grade (P=0.0001), and poor prognosis (P=0.0053). Suppression of IMP-1 expression with siRNA effectively suppressed growth of NSCLC cells. IMP-1 was able to bind to mRNAs encoding a variety of proteins involved in signal transduction, cell-cycle progression, cell adhesion and cytoskeleton, and various types of enzymatic activities. These results suggest that IMP-1 expression is likely to play important roles in lung cancer development and progression, and that IMP-1 is a prognostic marker and a promising therapeutic target for treatment of lung cancer.

This application claims the benefit of U.S. Provisional Application Ser. No. 60/838,750 filed Aug. 18, 2006, the contents of which are hereby incorporated by reference in their entirety.

### TECHNICAL FIELD

The present invention relates to methods for detecting, diagnosing, and prognosing cancer as well as methods for treating and preventing cancer.

### BACKGROUND ART

Lung cancer is one of the most common causes of cancer death worldwide, and non-small cell lung cancer (NSCLC) accounts for nearly 80% of those cases (Greenlee, R. T., et al. Cancer statistics, 2001. CA Cancer J Clin, 51: 15-36, 2001.). Regardless of histological subtype, the 5-year survival rate for lung-cancer patients hovers at about 10-15% (Jemal A, et al., (2004) CA Cancer J Clin; 54: 8-29. Naruke T, et al., (1998) J Thorac Cardiovasc Surg; 96: 440-7.). In fact, even those patients diagnosed at stage IA have a 5-year survival rate of less than 80% (Naruke T, et al., (1998) J Thorac Cardiovasc Surg; 96: 440-7. Chang M Y and Sugarbaker D J. (2003) Semin Surg Oncol; 21: 74-84.).

Many genetic alterations involved in development and progression of lung cancer have been reported; however, the precise molecular mechanisms remain unclear (Sozzi, G. Eur J Cancer, 37 Suppl 7: S63-73, 2001.). Over the last decade, newly developed cytotoxic agents, including paclitaxel, docetaxel, gemcitabine, and vinorelbine, have emerged to offer multiple therapeutic choices for patients with advanced NSCLC. However, those regimens provide only limited survival benefits compared with cisplatin-based therapies (Schiller, J. H., et al. N Engl J Med, 346: 92-98, 2002.; Kelly, K., et al. J Clin Oncol, 19: 3210-3218, 2001.). Recently developed agents targeting the EGFR pathway, such as erlotinib (Tarceva, OSI Pharmaceuticals) and gefitinib (Iressa, AstraZeneca), have been shown to be very effective to a subset of NSCLC patients. However, even if all kinds of available treatments are applied, the proportion of patients showing good response is still very limited (Lynch, T. J., et al. N Engl J Med, 350: 2129-2139, 2004.; Paez, J. G., et al. Science, 304: 1497-1500, 2004.; Tsao, M. S., et al. N Engl J Med, 353: 133-144, 2005.; Shepherd, F. A., et al. N Engl J Med, 353: 123-132, 2005.). Hence, new therapeutic strategies are eagerly anticipated.

Systematic analysis of expression levels of thousands of genes using cDNA microarray is an effective approach to identify molecules involved in carcinogenic pathways that can serve as candidates for development of novel therapeutics and diagnostics. In an attempt isolate potential molecular targets for the diagnosis and/or treatment of lung cancer, the present inventors have analyzed genome-wide expression profiles of various types of lung cancer cells on a cDNA microarray containing 27,648 genes, using tumor-cell populations purified by laser-capture microdissection (see WO2004/31413, incorporated by reference herein; see also Kikuchi, T., et al. Oncogene, 22: 2192-2205, 2003.; Kakiuchi S, et al. Hum Mol Genet, 13: 3029-3043, 2004.). To verify the biological and clinicopathological significance of the respective gene products, the present inventors have also performed tumor-tissue microarray analysis of clinical lung-cancer materials (Ishikawa, N., et al. Clin Cancer Res, 10: 8363-8370, 2004.; Kato, T., et al. Cancer Res, 65: 5638-5646, 2005.; Furukawa, C., et al. Cancer Res, 65: 7102-7110, 2005.; Suzuki, C., et al. Cancer Res, 65: 11314-11325, 2005.). Using this systematic approach, the present inventors discovered that IGF-II mRNA-binding protein 1 (IMP-1, alias CRDBP, c-myc coding region determinant binding protein, GenBank Accession No. NM_{—}006546, SEQ ID NO: 12 encoded by SEQ ID NO: 11) is frequently over-expressed in primary NSCLCs.

IMP-1 is a member of the ZBPs (zipcode-binding proteins) family, which includes orthologous and paralogous members of the same vertebrate RNA-binding protein family and consist of two RRMs (RNA recognition motifs) and four KH (K homology) domains (Nielsen, J., et al. Biochem J, 376: 383-391, 2003.). IMP-1 is expressed in most embryonic tissues. Analysis of total RNA from mouse embryos indicated peak IMP-1 expression at embryonic day 12.5, followed by decline toward birth and its disappearance in neonatal mice shortly after birth (Nielsen, J., et al. Mol Cell Biol, 19: 1262-1270, 1999.). IMP-1 is over-expressed in several human cancers, and has been suggested to play various roles in determining the post-transcriptional fate of its RNA targets and to act as a nucleocytoplasmic shuttling protein exhibiting a distinct pattern of localization in the cytoplasm (Nielsen, J., et al. Biochem J, 376: 383-391, 2003.; Ross, J., et al. Oncogene, 20: 6544-6550, 2001.; Ioannidis, P., et al. Int J Cancer, 104: 54-59, 2003.; Ioannidis, P., et al. Cancer Lett, 209: 245-250, 2004.; Gu, L., et al. Int J Oncol, 24: 671-678, 2004.; Nielsen, F. C., et al. J Cell Sci, 115: 2087-2097, 2002.; Runge, S., et al. J Biol Chem, 275: 29562-29569, 2000.). The protein is distributed along with microtubules and is likely to be transported toward the leading edge in motile cells. Its nuclear export and cytoplasmic movement depend on RNA binding, which implies that IMP-1 recognizes its targets in the nucleus and thereby defines their cytoplasmic fate. IMP-1 was indicated to play a significant role in polarizing genetic information by defining cytoplasmic RNA localization, an especially critical mechanism in developmental systems for the generation of subcellular asymmetries in protein abundance. H19 RNA co-localizes with IMP-1, and removal of the high-affinity attachment site leads to delocalization of the truncated RNA (Runge, S., et al. J Biol Chem, 275: 29562-29569, 2000.), which suggests that IMP-1 is involved in cytoplasmic trafficking of mRNA (Nielsen, F. C., et al. J Cell Sci, 115: 2087-2097, 2002.).

The present inventors herein report the identification of IMP-1 as a novel prognostic marker and a potential target for therapeutic agents, and also provide evidence for its possible role in human pulmonary carcinogenesis through its binding to various mRNAs which encode proteins related with cell proliferation and invasion.

Recent years, a new approach of cancer therapy using gene-specific siRNA was attempted in clinical trials (Bumcrot D et al., Nat Chem Biol 2006 December, 2(12): 711-9). RNAi seems to have already earned a place among the major technology platforms (Putral L N et al., Drug News Perspect 2006 July-August, 19(6): 317-24; Frantz S, Nat Rev Drug Discov 2006 July, 5(7): 528-9; Dykxhoorn D M et al., Gene Ther 2006 March, 13(6): 541-52). Nevertheless, there are several challenges that need to be faced before RNAi can be applied in clinical use. These challenges include poor stability of RNA in vivo (Hall A H et al., Nucleic Acids Res 2004 Nov. 15, 32(20): 5991-6000, Print 2004; Amarzguioui Metal., Nucleic Acids Res 2003 Jan. 15, 31(2): 589-95), toxicity as an agent (Frantz S, Nat Rev Drug Discov 2006 July, 5(7): 528-9), mode of delivery, the precise sequence of the siRNA or shRNA used, and cell type specificity. It is well-known fact that there are possible toxicities related to silencing of partially homologous genes or induction of universal gene suppression by activating the interferon response (Judge A D et al., Nat Biotechnol 2005 April, 23(4): 457-62, Epub 2005 Mar. 20; Jackson A L & Linsley P S, Trends Genet 2004 November, 20(11): 521-4). So double-stranded molecules targeting cancer-specific genes, which molecules are devoid of adverse side-effects, are needed for the development of anticancer drugs.

### DISCLOSURE OF THE INVENTION

The present invention is based on the discovery of a specific expression pattern of the IMP-1 gene in cancerous cells.

Through an analysis on genome-wide expression profiles of genes in various types of lung cancer cells, a set of genes whose expression was commonly up-regulated was identified. From among the genes, the present inventors selected gene IMP-1 (an IGF-II mRNA-binding protein 1) for further study. The expression of the IMP-1 gene was detected by the present inventors to be enhanced in lung carcinomas. In the course of the present invention, the IMP-1 gene was further revealed to be frequently up-regulated in non-small cell lung cancer (NSCLC), including adenocarcinomas (ADCs), squamous-cell carcinomas (SCCs) and small-cell lung cancer (SCLC). Furthermore, the protein encoded by the gene was discovered to play various roles in determining the post-transcriptional fate of its RNA targets and to act as a nucleocytoplasmic shuttling protein exhibiting a distinct pattern of localization in the cytoplasm (Nielsen, J., et al. Biochem J, 376: 383-391, 2003.; Ross, J., et al. Oncogene, 20: 6544-6550, 2001.; Ioannidis, P., et al. Int J Cancer, 104: 54-59, 2003.; Ioannidis, P., et al. Cancer Lett, 209: 245-250, 2004.; Gu, L., et al. Int J Oncol, 24: 671-678, 2004.; Nielsen, F. C., et al. J Cell Sci, 115: 2087-2097, 2002.; Runge, S., et al. J Biol Chem, 275: 29562-29569, 2000.). Moreover, since the suppression of the IMP-1 gene by small interfering RNA (siRNA) resulted in growth inhibition and/or cell death of NSCLC cells, this gene may serve as a novel therapeutic target for various types of human neoplasms.

The IMP-1 gene identified herein, as well as its transcription and translation products, finds diagnostic utility as a marker for cancer and as an oncogene target, the expression and/or activity of which may be altered to treat or alleviate a symptom of cancer. Similarly, by detecting the changes in the expression of the IMP-1 gene due to a compound, various compounds can be identified as agents for treating or preventing cancer.

Accordingly, the present invention provides a method for diagnosing or determining a predisposition to cancer in a subject by determining the expression level of the IMP-1 gene in a subject-derived biological sample, such as tissue sample. Increased expression level of the gene as compared to a normal control level indicates that the subject suffers from or is at risk of developing cancer. The normal control level can be determined using a normal cell obtained from a non-cancerous tissue.

In the present invention, preferred cancer is NSCLC.

In the context of the present invention, the phrase “control level” refers to the expression level of the IMP-1 gene detected in a control sample and includes both normal control level and cancer control level. A control level can be a single expression pattern derived from a single reference population or from a plurality of expression patterns. For example, the control level can be a database of expression patterns from previously tested cells. A “normal control level” refers to a level of the IMP-1 gene expression detected in a normal healthy individual or in a population of individuals known not to be suffering from cancer. A normal individual is one with no clinical symptom of cancer. On the other hand, a “cancer control level” refers to an expression level of the IMP-1 gene found in an individual or population suffering from cancer.

An increase in the expression level of the IMP-1 gene detected in a sample as compared to a normal control level indicates that the subject (from which the sample has been obtained) suffers from or is at risk of cancer.

Alternatively, expression levels of a panel of genes including the IMP-1 gene in a sample can be compared to cancer control levels of the same panel of genes. A similarity between the expression levels of a sample and the cancer control levels indicates that the subject (from which the sample has been obtained) suffers from or is at risk of cancer.

Herein, gene expression levels are deemed to be “altered” when the gene expression increases or decreases by, for example, 10%, 25%, or 50% from, or at least 0.1 fold, at least 0.2 fold, at least 1 fold, at least 2 fold, at least 5 fold, or at least 10 fold or more compared to a control level. The expression level of the IMP-1 gene can be determined by detecting, e.g., hybridization intensity of nucleic acid probes to gene transcripts in a sample.

In the context of the present invention, subject-derived tissue samples may be any tissues obtained from test subjects, e.g., patients known to have or suspected of having cancer. For example, tissues may include epithelial cells. More particularly, tissues may be cancerous epithelial cells.

Herein, evidence is presented that IMP-1 over-expression is associated with lung cancer progression, resulting in a poor prognosis for patients with lung cancer. Thus, the IMP-1 gene may serve as a useful prognostic indicator of lung cancer. In particular, IMP-1 over-expression in resected specimens may be a useful index for application of adjuvant therapy to the patients who are likely to have poor prognosis. Furthermore, in that up-regulation of IMP-1 is a frequent and important feature of lung carcinogenesis, the present inventors accordingly propose that targeting the IMP-1 molecule holds promise for development of new diagnostic strategies for clinical management of lung cancers.

Accordingly, it is an object of the present invention to provide a method for assessing or determining the prognosis of a patient with non-small cell lung cancer by comparing an IMP-1 level in a patient-derived biological sample with that of a control sample. An elevated expression level is indicative of a poor prognosis for post-treatment remission, recovery and/or survival and a higher likelihood of poor clinical outcome. It is a further object of the present invention to provide kits for assessing an NSCLC prognosis, such kits including IMP-1-detection reagents.

The present invention further provides methods for identifying compounds that inhibit or enhance the expression or activity of IMP-1, by contacting a test cell expressing IMP-1 with test compounds and determining the expression level of the IMP-1 gene or the activity of the gene product. The test cell may be an epithelial cell, such as cancerous epithelial cell. A decrease in the expression level of the gene or the activity of its gene product as compared to a control level in the absence of the test compound indicates that the test compound may be used to reduce symptoms of cancer.

Therapeutic methods of the present invention include methods for treating or preventing cancer in a subject including the step of administering an antisense composition to the subject. In the context of the present invention, the antisense composition reduces the expressions of a specific target gene (i.e., the IMP-1 gene). For example, the antisense compositions may contain a nucleotide which is complementary to the IMP-1 gene sequence. Alternatively, the present methods may include the step of administering an siRNA composition to the subject. In the context of the present invention, the siRNA composition reduces the expression of the IMP-1 gene. In yet another method, the treatment or prevention of cancer in a subject may be carried out by administering a ribozyme composition to the subject. In the context of the present invention, the nucleic acid-specific ribozyme composition reduces the expression of the IMP-1 gene. In fact, the present inventors confirmed inhibitory effects of siRNAs for the IMP-1 gene. For example, the inhibition of cell proliferation of cancer cells by the siRNAs are demonstrated in the Examples section, which supports the fact that the IMP-1 gene serves as a preferable therapeutic target for cancer.

One advantage of the methods described herein is that the disease is identified prior to detection of overt clinical symptoms of cancers. Other features and advantages of the invention will be apparent from the following detailed description, and from the claims. However, it is to be understood that both the foregoing summary of the invention and the following detailed description are of a preferred embodiment, and not restrictive of the invention or other alternate embodiments of the invention.

### BRIEF DESCRIPTION OF THE DRAWINGS

FIG. 1 depicts the expression of IMP-1 in lung tumors and normal tissues. Part A: Expression of IMP-1 in clinical samples of NSCLC (T) and corresponding normal lung tissues (N), examined by semiquantitative RT-PCR. Expression of β-actin (ACTB) was served as a quantity control. Part B: Expression of IMP-1 transcripts in lung-cancer cell lines, as revealed by semiquantitative RT-PCR. Part C: Specificity of anti-IMP-1 antibody displaying reaction with only IMP-1 protein, but no cross-reaction with other homologous proteins, IMP-2 and IMP-3 using lysates from NCI-H520 cells transfected with IMP-1, -2, and -3 expressing vector (left panels). Expression of IMP-1 protein in lung-cancer cell lines by western blot analysis (right panels). Expression of ACTB was served as a quantity control. Part D: Expression of IMP-1 in normal human tissues, detected by northern-blot analysis.

FIG. 2 depicts the association of IMP-1 over-expression with poor prognosis of NSCLC patients. Part A: Representative example of positive- or negative-expression of IMP-1 in lung cancer (SCC, ×100) and normal lung. Detection of IMP-1 protein by immunohistochemistry using the rabbit polyclonal anti-IMP-1 antibody; counterstaining with Hematoxylin. Part B: Magnified view (SCC, ×200). Part C: Kaplan-Meier analysis of tumor specific survival in NSCLC patients according to IMP-1 expression level.

FIG. 3 depicts the inhibition of growth of NSCLC cells by siRNA against IMP-1. Part A: Response of A549 cells to si-IMP-1 or control siRNAs (si-EGFP or si-Scramble). The level of IMP-1 expression detected by semiquantitative RT-PCR in cells treated with either control or si-IMP-1s is shown in the upper panels. Colony-formation assays using A549 cells transfected with siRNA to IMP-1 (#1-#3) is shown in lower panels. Part B: The effect of siRNA against IMP-1 on cell viability, detected by MTT assays.

### DETAILED DESCRIPTION OF THE INVENTION

The words “a”, “an”, and “the” as used herein mean “at least one” unless otherwise specifically indicated.

The terms “isolated” and “purified” when used herein in relation to a substance (e.g., polypeptide, antibody, polynucleotide, etc.) indicate that the substance is substantially free from at least one substance that may else be included in the natural source. Thus, an isolated or purified antibody refers to antibodies that is substantially free of cellular material such as carbohydrate, lipid, or other contaminating proteins from the cell or tissue source from which the protein (antibody) is derived, or substantially free of chemical precursors or other chemicals when chemically synthesized. The term “substantially free of cellular material” includes preparations of a polypeptide in which the polypeptide is separated from cellular components of the cells from which it is isolated or recombinantly produced. Thus, a polypeptide that is substantially free of cellular material includes preparations of polypeptide having less than about 30%, 20%, 10%, or 5% (by dry weight) of heterologous protein (also referred to herein as a “contaminating protein”). When the polypeptide is recombinantly produced, it is also preferably substantially free of culture medium, which includes preparations of polypeptide with culture medium less than about 20%, 10%, or 5% of the volume of the protein preparation. When the polypeptide is produced by chemical synthesis, it is preferably substantially free of chemical precursors or other chemicals, which includes preparations of polypeptide with chemical precursors or other chemicals involved in the synthesis of the protein less than about 30%, 20%, 10%, 5% (by dry weight) of the volume of the protein preparation. That a particular protein preparation contains an isolated or purified polypeptide can be shown, for example, by the appearance of a single band following sodium dodecyl sulfate (SDS)-polyacrylamide gel electrophoresis of the protein preparation and Coomassie Brilliant Blue staining or the like of the gel. In a preferred embodiment, antibodies of the present invention are isolated or purified.

An “isolated” or “purified” nucleic acid molecule, such as a cDNA molecule, can be substantially free of other cellular material, or culture medium when produced by recombinant techniques, or substantially free of chemical precursors or other chemicals when chemically synthesized. In a preferred embodiment, nucleic acid molecules encoding antibodies of the present invention are isolated or purified.

The terms “polypeptide”, “peptide”, and “protein” are used interchangeably herein to refer to a polymer of amino acid residues. The terms apply to amino acid polymers in which one or more amino acid residue is a modified residue, or a non-naturally occurring residue, such as an artificial chemical mimetic of a corresponding naturally occurring amino acid, as well as to naturally occurring amino acid polymers.

The term “amino acid” refers to naturally occurring and synthetic amino acids, as well as amino acid analogs and amino acid mimetics that similarly functions to the naturally occurring amino acids. Naturally occurring amino acids are those encoded by the genetic code, as well as those modified after translation in cells (e.g., hydroxyproline, γ-carboxyglutamate, and O-phosphoserine). The phrase “amino acid analog” refers to compounds that have the same basic chemical structure (an α carbon bound to a hydrogen, a carboxy group, an amino group, and an R group) as a naturally occurring amino acid but have a modified R group or modified backbones (e.g., homoserine, norleucine, methionine, sulfoxide, methionine methyl sulfonium). The phrase “amino acid mimetic” refers to chemical compounds that have different structures but similar functions to general amino acids.

Amino acids may be referred to herein by their commonly known three letter symbols or the one-letter symbols recommended by the IUPAC-IUB Biochemical Nomenclature Commission.

The terms “polynucleotides”, “oligonucleotide”, “nucleotides”, “nucleic acids”, and “nucleic acid molecules” are used interchangeably unless otherwise specifically indicated and, similarly to the amino acids, are referred to by their commonly accepted single-letter codes. Similar to the amino acids, they encompass both naturally-occurring and non-naturally occurring nucleic acid polymers. The polynucleotide, oligonucleotide, nucleotides, nucleic acids, or nucleic acid molecules may be composed of DNA, RNA or a combination thereof.

The present invention is based in part on the discovery of elevated expression of the IMP-1 gene in cells from patients of lung cancers. The nucleotide sequence of the human IMP-1 gene is shown in SEQ ID NO: 11 and is also available as GenBank Accession No. NM_{—}006546. Herein, the IMP-1 gene encompasses the human IMP-1 gene as well as those of other animals, including non-human primate, mouse, rat, dog, cat, horse, and cow. However, the invention is not limited thereto and includes allelic mutants and genes found in other animals as corresponding to the IMP-1 gene.

The amino acid sequence encoded by the human IMP-1 gene is shown in SEQ ID NO: 12 and is also available as GenBank Accession No. NP_{—}006537. In the present invention, the polypeptide encoded by the IMP-1 gene is referred to as “IMP-1”, and sometimes as “IMP-1 polypeptide” or “IMP-1 protein”.

According to an aspect of the present invention, functional equivalents are also considered to be “IMP-1 polypeptides”. Herein, a “functional equivalent” of a protein is a polypeptide that has a biological activity equivalent to the protein. Namely, any polypeptide that retains the biological ability of the IMP-1 protein may be used as such a functional equivalent in the present invention. Such functional equivalents include those wherein one or more amino acids are substituted, deleted, added, or inserted to the natural occurring amino acid sequence of the IMP-1 protein. Alternatively, the polypeptide may be composed an amino acid sequence having at least about 80% homology (also referred to as sequence identity) to the sequence of the respective protein, more preferably at least about 90% to 95% homology. In other embodiments, the polypeptide can be encoded by a polynucleotide that hybridizes under stringent conditions to the natural occurring nucleotide sequence of the IMP-1 gene.

A polypeptide of the present invention may have variations in amino acid sequence, molecular weight, isoelectric point, the presence or absence of sugar chains, or form, depending on the cell or host used to produce it or the purification method utilized. Nevertheless, so long as it has a function equivalent to that of the human IMP-1 protein of the present invention, it is within the scope of the present invention.

The phrase “stringent (hybridization) conditions” refers to conditions under which a nucleic acid molecule will hybridize to its target sequence, typically in a complex mixture of nucleic acids, but not detectably to other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times of background, preferably 10 times of background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 50° C.

In the context of the present invention, a condition of hybridization for isolating a DNA encoding a polypeptide functionally equivalent to the human IMP-1 protein can be routinely selected by a person skilled in the art. For example, hybridization may be performed by conducting pre-hybridization at 68° C. for 30 min or longer using “Rapid-hyb buffer” (Amersham LIFE SCIENCE), adding a labeled probe, and warming at 68° C. for 1 hour or longer. The following washing step can be conducted, for example, in a low stringent condition. An exemplary low stringent condition may include 42° C., 2×SSC, 0.1% SDS, preferably 50° C., 2×SSC, 0.1% SDS. High stringency conditions are often preferably used. An exemplary high stringency condition may include washing 3 times in 2×SSC, 0.01% SDS at room temperature for 20 min, then washing 3 times in 1×SSC, 0.1% SDS at 37° C. for 20 min, and washing twice in 1×SSC, 0.1% SDS at 50° C. for 20 min. However, several factors, such as temperature and salt concentration, can influence the stringency of hybridization and one skilled in the art can suitably select the factors to achieve the requisite stringency.

Generally, it is known that modifications of one or more amino acid in a protein do not influence the function of the protein. In fact, mutated or modified proteins, proteins having amino acid sequences modified by substituting, deleting, inserting, and/or adding one or more amino acid residues of a certain amino acid sequence, have been known to retain the original biological activity (Mark et al., Proc Natl Acad Sci USA 81: 5662-6 (1984); Zoller and Smith, Nucleic Acids Res 10:6487-500 (1982); Dalbadie-McFarland et al., Proc Natl Acad Sci USA 79: 6409-13 (1982)). Accordingly, one of skill in the art will recognize that individual additions, deletions, insertions, or substitutions to an amino acid sequence which alter a single amino acid or a small percentage of amino acids or those considered to be a “conservative modifications”, wherein the alteration of a protein results in a protein with similar functions, are acceptable in the context of the instant invention.

So long as the activity the protein is maintained, the number of amino acid mutations is not particularly limited. However, it is generally preferred to alter 5% or less of the amino acid sequence. Accordingly, in a preferred embodiment, the number of amino acids to be mutated in such a mutant is generally 30 amino acids or less, preferably 20 amino acids or less, more preferably 10 amino acids or less, more preferably 6 amino acids or less, and even more preferably 3 amino acids or less.

An amino acid residue to be mutated is preferably mutated into a different amino acid in which the properties of the amino acid side-chain are conserved (a process known as conservative amino acid substitution). Examples of properties of amino acid side chains are hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). Conservative substitution tables providing functionally similar amino acids are well known in the art. For example, the following eight groups each contain amino acids that are conservative substitutions for one another:

1) Alanine (A), Glycine (G);

2) Aspartic acid (D), Glutamic acid (E);

3) Aspargine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cystein (C), Methionine (M) (see, e.g., Creighton, Proteins 1984).

Such conservatively modified polypeptides are included in the present IMP-1 protein. However, the present invention is not restricted thereto and the IMP-1 protein includes non-conservative modifications, so long as at least one biological activity of the IMP-1 protein is retained. Furthermore, the modified proteins do not exclude polymorphic variants, interspecies homologues, and those encoded by alleles of these proteins.

Moreover, the IMP-1 gene of the present invention encompasses polynucleotides that encode such functional equivalents of the IMP-1 protein. In addition to hybridization, a gene amplification method, for example, the polymerase chain reaction (PCR) method, can be utilized to isolate a polynucleotide encoding a polypeptide functionally equivalent to the IMP-1 protein, using a primer synthesized based on the sequence information of the protein encoding DNA (SEQ ID NO: 12). Polynucleotides and polypeptides that are functionally equivalent to the human IMP-1 gene and protein, respectively, normally have a high homology to the originating nucleotide or amino acid sequence of. “High homology” typically refers to a homology of 40% or higher, preferably 60% or higher, more preferably 80% or higher, even more preferably 90% to 95% or higher. The homology of a particular polynucleotide or polypeptide can be determined by following the algorithm in “Wilbur and Lipman, Proc Natl Acad Sci USA 80: 726-30 (1983)”.

### I. Double-Stranded Molecule:

As use herein, the term “double-stranded molecule” refers to a nucleic acid molecule that inhibits expression of a target gene including, for example, short interfering RNA (siRNA; e.g., double-stranded ribonucleic acid (dsRNA) or small hairpin RNA (shRNA)) and short interfering DNA/RNA (siD/R-NA; e.g. double-stranded chimera of DNA and RNA (dsD/R-NA) or small hairpin chimera of DNA and RNA (shD/R-NA)).

As used herein, the term “dsRNA” refers to a construct of two RNA molecules comprising complementary sequences to one another and that have annealed together via the complementary sequences to form a double-stranded RNA molecule. The nucleotide sequence of two strands may comprise not only the “sense” or “antisense” RNAs selected from a protein coding sequence of target gene sequence, but also RNA molecule having a nucleotide sequence selected from non-coding region of the target gene.

The term “shRNA”, as used herein, refers to an siRNA having a stem-loop structure, comprising a first and second regions complementary to one another, i.e., sense and antisense strands. The degree of complementarity and orientation of the regions being sufficient such that base pairing occurs between the regions, the first and second regions being joined by a loop region, the loop resulting from a lack of base pairing between nucleotides (or nucleotide analogs) within the loop region. The loop region of an shRNA is a single-stranded region intervening between the sense and antisense strands and may also be referred to as “intervening single-strand”.

As use herein, the term “siD/R-NA” refers to a double-stranded polynucleotide molecule which is composed of both RNA and DNA, and includes hybrids and chimeras of RNA and DNA and prevents translation of a target mRNA. Herein, a hybrid indicates a molecule wherein a polynucleotide composed of DNA and a polynucleotide composed of RNA hybridize to each other to form the double-stranded molecule; whereas a chimera indicates that one or both of the strands composing the double stranded molecule may contain RNA and DNA. Standard techniques of introducing siD/R-NA into the cell are used. The siD/R-NA includes a sense nucleic acid sequence (also referred to as “sense strand”), an antisense nucleic acid sequence (also referred to as “antisense strand”) or both. The siD/R-NA may be constructed such that a single transcript has both the sense and complementary antisense nucleic acid sequences from the target gene, e.g., a hairpin. The siD/R-NA may either be a dsD/R-NA or shD/R-NA.

As used herein, the term “dsD/R-NA” refers to a construct of two molecules comprising complementary sequences to one another and that have annealed together via the complementary sequences to form a double-stranded polynucleotide molecule. The nucleotide sequence of two strands may comprise not only the “sense” or “antisense” polynucleotides sequence selected from a protein coding sequence of target gene sequence, but also polynucleotide having a nucleotide sequence selected from non-coding region of the target gene. One or both of the two molecules constructing the dsD/R-NA are composed of both RNA and DNA (chimeric molecule), or alternatively, one of the molecules is composed of RNA and the other is composed of DNA (hybrid double-strand).

The term “shD/R-NA”, as used herein, refers to an siD/R-NA having a stem-loop structure, comprising a first and second regions complementary to one another, i.e., sense and antisense strands. The degree of complementarity and orientation of the regions being sufficient such that base pairing occurs between the regions, the first and second regions being joined by a loop region, the loop resulting from a lack of base pairing between nucleotides (or nucleotide analogs) within the loop region. The loop region of an shD/R-NA is a single-stranded region intervening between the sense and antisense strands and may also be referred to as “intervening single-strand”.

The double-stranded molecules of the invention may contain one or more modified nucleotides and/or non-phosphodiester linkages. Chemical modifications well known in the art are capable of increasing stability, availability, and/or cell uptake of the double-stranded molecule. The skilled person will be aware of other types of chemical modification which may be incorporated into the present molecules (WO03/070744; WO2005/045037). In one embodiment, modifications can be used to provide improved resistance to degradation or improved uptake. Examples of such modifications include phosphorothioate linkages, 2′-O-methyl ribonucleotides (especially on the sense strand of a double-stranded molecule), 2′-deoxy-fluoro ribonucleotides, 2′-deoxy ribonucleotides, “universal base” nucleotides, 5′-C-methyl nucleotides, and inverted deoxyabasic residue incorporation (US20060122137).

In another embodiment, modifications can be used to enhance the stability or to increase targeting efficiency of the double-stranded molecule. Modifications include chemical cross linking between the two complementary strands of a double-stranded molecule, chemical modification of a 3′ or 5′ terminus of a strand of a double-stranded molecule, sugar modifications, nucleobase modifications and/or backbone modifications, 2-fluoro modified ribonucleotides and 2′-deoxy ribonucleotides (WO2004/029212). In another embodiment, modifications can be used to increased or decreased affinity for the complementary nucleotides in the target mRNA and/or in the complementary double-stranded molecule strand (WO2005/044976). For example, an unmodified pyrimidine nucleotide can be substituted for a 2-thio, 5-alkynyl, 5-methyl, or 5-propynyl pyrimidine. Additionally, an unmodified purine can be substituted with a 7-deza, 7-alkyl, or 7-alkenyi purine. In another embodiment, when the double-stranded molecule is a double-stranded molecule with a 3′ overhang, the 3′-terminal nucleotide overhanging nucleotides may be replaced by deoxyribonucleotides (Elbashir S M et al., Genes Dev 2001 Jan. 15, 15(2): 188-200). For further details, published documents such as US20060234970 are available. The present invention is not limited to these examples and any known chemical modifications may be employed for the double-stranded molecules of the present invention so long as the resulting molecule retains the ability to inhibit the expression of the target gene.

Furthermore, the double-stranded molecules of the invention may comprise both DNA and RNA, e.g., dsD/R-NA or shD/R-NA. Specifically, a hybrid polynucleotide of a DNA strand and an RNA strand or a DNA-RNA chimera polynucleotide shows increased stability. Mixing of DNA and RNA, i.e., a hybrid type double-stranded molecule consisting of a DNA strand (polynucleotide) and an RNA strand (polynucleotide), a chimera type double-stranded molecule comprising both DNA and RNA on any or both of the single strands (polynucleotides), or the like may be formed for enhancing stability of the double-stranded molecule. The hybrid of a DNA strand and an RNA strand may be the hybrid in which either the sense strand is DNA and the antisense strand is RNA, or the opposite so long as it has an activity to inhibit expression of the target gene when introduced into a cell expressing the gene. Preferably, the sense strand polynucleotide is DNA and the antisense strand polynucleotide is RNA. Also, the chimera type double-stranded molecule may be either where both of the sense and antisense strands are composed of DNA and RNA, or where any one of the sense and antisense strands is composed of DNA and RNA so long as it has an activity to inhibit expression of the target gene when introduced into a cell expressing the gene.

In order to enhance stability of the double-stranded molecule, the molecule preferably contains as much DNA as possible, whereas to induce inhibition of the target gene expression, the molecule is required to be RNA within a range to induce sufficient inhibition of the expression. As a preferred example of the chimera type double-stranded molecule, an upstream partial region (i.e., a region flanking to the target sequence or complementary sequence thereof within the sense or antisense strands) of the double-stranded molecule is RNA. Preferably, the upstream partial region indicates the 5′ side (5′-end) of the sense strand and the 3′ side (3′-end) of the antisense strand. That is, in preferable embodiments, a region flanking to the 3′-end of the antisense strand, or both of a region flanking to the 5′-end of sense strand and a region flanking to the 3′-end of antisense strand consists of RNA. For instance, the chimera or hybrid type double-stranded molecule of the present invention comprise following combinations.

| | | |
|---|---|---|
| sense strand: | 5′-[DNA]-3′ | |
| | 3′-(RNA)-[DNA]-5′ | antisense strand, |
| sense strand: | 5′-(RNA)-[DNA]-3′ | |
| | 3′-(RNA)-[DNA]-5′ | antisense strand, |
| and | | |
| sense strand: | 5′-(RNA)-[DNA]-3′ | |
| | 3′-(RNA)-5′ | antisense strand. |

The upstream partial region preferably is a domain consisting of 9 to 13 nucleotides counted from the terminus of the target sequence or complementary sequence thereto within the sense or antisense strands of the double-stranded molecules. Moreover, preferred examples of such chimera type double-stranded molecules include those having a strand length of 19 to 21 nucleotides in which at least the upstream half region (5′ side region for the sense strand and 3′ side region for the antisense strand) of the polynucleotide is RNA and the other half is DNA. In such a chimera type double-stranded molecule, the effect to inhibit expression of the target gene is much higher when the entire antisense strand is RNA (US20050004064).

In the present invention, the double-stranded molecule may form a hairpin, such as a short hairpin RNA (shRNA) and short hairpin consisting of DNA and RNA (shD/R-NA). The shRNA or shD/R-NA is a sequence of RNA or mixture of RNA and DNA making a tight hairpin turn that can be used to silence gene expression via RNA interference. The shRNA or shD/R-NA comprises the sense target sequence and the antisense target sequence on a single strand wherein the sequences are separated by a loop sequence. Generally, the hairpin structure is cleaved by the cellular machinery into dsRNA or dsD/R-NA, which is then bound to the RNA-induced silencing complex (RISC). This complex binds to and cleaves mRNAs which match the target sequence of the dsRNA or dsD/R-NA.

Unless otherwise defined, all technical and scientific terms used herein have the same meaning as commonly understood by one of ordinary skill in the art to which this invention belongs. In case of conflict, the present specification, including definitions, will control.

A double-stranded molecule against the IMP-1 gene (e.g. ‘IMP-1 siRNA’) can be used to reduce the expression level of the gene. Herein, the term “siRNA” refers to a double-stranded RNA molecule which prevents translation of a target mRNA. In the context of the present invention, the double-stranded molecule is composed of a sense nucleic acid sequence and an anti-sense nucleic acid sequence against the up-regulated marker gene, IMP-1. The double-stranded molecule is constructed so that it includes both a sense and complementary antisense sequences of the target gene, i.e., a nucleotide having a hairpin structure. The double-stranded molecule may either be a dsRNA, shRNA, ds D/RNA or shD/RNA.

A double-stranded molecule of the IMP-1 gene hybridizes to target mRNA, i.e., associates with the normally single-stranded mRNA transcript and thereby interfering with translation of the mRNA, which finally decreases or inhibits production (expression) of the polypeptide encoded by the gene. Thus, an siRNA molecule of the invention can be defined by its ability to specifically hybridize to the mRNA of the IMP-1 gene under stringent conditions.

In the context of the present invention, a double-stranded molecule is preferably less than 500, 200, 100, 50, or 25 nucleotides in length. More preferably a double-stranded molecule is 19-25 nucleotides in length. Exemplary target nucleic acid sequences of IMP-1 double-stranded molecule include the oligonucleotide sequences corresponding to SEQ ID NO: 9 or 10. The nucleotide “t” in the sequence should be replaced with “u” in RNA or derivatives thereof. Accordingly, for example, the present invention provides double-stranded RNA molecules having the oligonucleotide sequence 5′-ggaggagaacuucuuuggu-3′ (SEQ ID NO: 9) or 5′-gaaucuauggcaaacucaa-3′ (SEQ ID NO: 10). In order to enhance the inhibition activity of the double-stranded molecules, nucleotide “u” can be added to the 3′ end of the antisense strand. The number of “u”s to be added is at least 2, generally 2 to 10, preferably 2 to 5. The added “u”s form a single strand at the 3′ end of the antisense strand of the double-stranded molecule.

A loop sequence composed of an arbitrary nucleotide sequence can be located between the sense and antisense sequence in order to form the hairpin loop structure. Thus, the present invention also provides double-stranded molecule having the general formula 5′-[A]-[B]-[A′]-3′, wherein [A] is a oligonucleotide sequence corresponding to a sequence that specifically hybridizes to an mRNA or a cDNA of the IMP-1 gene. In preferred embodiments, [A] is a ribonucleotide sequence corresponding to a sequence of the IMP-1 gene; [B] is a ribonucleotide sequence composed of 3 to 23 nucleotides; and [A′] is a ribonucleotide sequence composed of the complementary sequence of [A]. The region [A] hybridizes to [A′], and then a loop composed of region [B] is formed. The loop sequence may be preferably 3 to 23 nucleotide in length. The loop sequence, for example, can be selected from a group composed of following sequences (http://www.ambion.com/techlib/tb/tb_{—}506.html):

CCC, CCACC, or CCACACC: Jacque J M et al., Nature 2002, 418: 435-8.

UUCG: Lee N S et al., Nature Biotechnology 2002, 20:500-5; Fruscoloni P et al., Proc Natl Acad Sci USA 2003, 100(4):1639-44.

UUCAAGAGA: Dykxhoorn D M et al., Nature Reviews Molecular Cell Biology 2003, 4:457-67.

‘UUCAAGAGA (“ttcaagaga” in DNA)’ is a particularly suitable loop sequence. Furthermore, loop sequence consisting of 23 nucleotides also provides an active siRNA (Jacque J-M et al., Nature 2002, 418:435-8).

Exemplary hairpin double-stranded molecule suitable for use in the context of the present invention include,

5′-ggaggagaacuucuuuggu-[b]-accaaagaaguucuccucc-3′
(for target sequence of SEQ ID NO: 9);
and
5′-gaaucuauggcaaacucaa-[b]-uugaguuugccauagauuc-3′
(for target sequence of SEQ ID NO: 10).

The oligonucleotide sequence of suitable double-stranded molecules can be designed using an siRNA design computer program available from the Ambion website (http://www.ambion.com/techlib/misc/siRNA_finder.html). The computer program selects nucleotide sequences for double-stranded molecule synthesis based on the following protocol.

Selection of siRNA Target Sites:
1. Beginning with the AUG start codon of the object transcript, scan downstream for AA dinucleotide sequences. Record the occurrence of each AA and the 3′ adjacent 19 nucleotides as potential target sites. Tuschl et al. Genes Cev 1999, 13(24):3191-7 don't recommend against designing target sequence to the 5′ and 3′ untranslated regions (UTRs) and regions near the start codon (within 75 nucleotides) as these may be richer in regulatory protein binding sites. UTR-binding proteins and/or translation initiation complexes may interfere with binding of the endonuclease complex.
2. Compare the potential target sites to the human genome database and eliminate from consideration any target sequences with significant homology to other coding sequences. The homology search can be performed using BLAST (Altschul S F et al., Nucleic Acids Res 1997, 25:3389-402; J Mol Biol 1990, 215:403-10.), which can be found on the NCBI server at: www.ncbi.nlm.nih.gov/BLAST/.
3. Select qualifying target sequences for synthesis. At Ambion, preferably several target sequences can be selected along the length of the gene to evaluate.

Standard techniques for introducing a double-stranded molecule into the cell may be used. For example, a double-stranded molecule of IMP-1 can be directly introduced into the cells in a form that is capable of binding to the mRNA transcripts. In these embodiments, the double-stranded molecules of the present invention are typically modified as described above for antisense molecules. Other modifications are also possible, for example, cholesterol-conjugated double-stranded molecules have shown improved pharmacological properties (Song et al., Nature Med 2003, 9:347-51).

Alternatively, a DNA encoding the double-stranded molecule may be carried in a vector (hereinafter, also referred to as ‘siRNA vector’). Such vectors may be produced, for example, by cloning the target IMP-1 gene sequence into an expression vector having operatively-linked regulatory sequences (e.g., a RNA polymerase III transcription unit from the small nuclear RNA (snRNA) U6 or the human H1 RNA promoter) flanking the sequence in a manner that allows for expression (by transcription of the DNA molecule) of both strands (Lee N S et al., Nature Biotechnology 2002, 20: 500-5). For example, an RNA molecule that is antisense to mRNA of the IMP-1 gene is transcribed by a first promoter (e.g., a promoter sequence 3′ of the cloned DNA) and an RNA molecule that is the sense strand for the mRNA of the IMP-1 gene is transcribed by a second promoter (e.g., a promoter sequence 5′ of the cloned DNA). The sense and antisense strands hybridize in vivo to generate double-stranded molecule constructs for silencing the expression of the IMP-1 gene. Alternatively, the two constructs can be utilized to create the sense and anti-sense strands of a single-stranded construct. In this case, a construct having secondary structure, e.g., hairpin, is produced as a single transcript that includes both the sense and complementary antisense sequences of the target gene.

For introducing the vector of double-stranded molecule into the cell, transfection-enhancing agent can be used. FuGENE6 (Roche diagnostics), Lipofectamine 2000 (Invitrogen), Oligofectamine (Invitrogen), and Nucleofector (Wako pure Chemical) are useful as the transfection-enhancing agent. Therefore, the present pharmaceutical composition may further include such transfection-enhancing agents.

### II. Antibody:

The present invention provides antibodies against an IMP-1 protein but not IMP-2 and IMP-3, or fragments of the antibodies. In other words, the antibodies of the present invention can be used for detecting an IMP-1 specific expression. Therefore, the antibodies of the present invention are useful for diagnosing IMP-1 related diseases, for example lung cancer, e.g. NSCLC and treating those diseases. The antibody can be prepared by using IMP-1 fragments that not identical to IMP-2 and IMP-3, e.g. an amino acid sequence set forth in SEQ ID NO: 5 or SEQ ID NO: 6, see the item of ‘D. Preparation of anti-IMP-1 polyclonal antibody’ in EXAMPLE.

When the expression of IMP-1 is observed by tissue immunostaining, the survival rate is low in the patient with lung cancer, as shown in Table 1. This finding suggests that the expression of IMP-1 should be useful in diagnosing malignant prognosis as an index. However, it is not found that IMP-2 and IMP-3 are correlated with malignant prognosis. Therefore, prognosis may be diagnosed more accurately using the IMP-1 specific antibody.

Furthermore, although IMP-1, IMP-2 and IMP-3 are all lung cancer related genes as shown in our previous application (WO 2004/031413), the results of RT-PCR in the previous application showed that the expression patterns of these genes are different from each other, indicating that highly accurate diagnosis may be achieved by using the IMP-1 specific antibody of the present invention.

Alternatively, since each of IMP-1, IMP-2 and IMP-3 is translated from a different gene, IMP-1 specific agents (e.g. IMP-1 specific siRNAs or antibodies) can be effective, selectively in cases where IMP-1 is over-expressed. Specifically, even through IMP-2 or IMP-3 is over-expressed, IMP-1 specific agents may have lower or no effect when the expression of IMP-1 is suppressed. Therefore, for selecting appropriate agents, it is required to determine whether IMP-1 is expressed in the focal tissue. The antibody of the present invention may be a useful tool for such detection of IMP-1 prior to administering such agents.

Furthermore, the antibody of the present invention may be a useful tool for functional analysis of IMP-1.

Furthermore, the antibody of the present invention must be an useful tool for functional analysis of IMP-1. The term “antibody” as used herein encompasses naturally occurring antibodies as well as non-naturally occurring antibodies, including, for example, single chain antibodies, chimeric, bifunctional and humanized antibodies, as well as antigen-binding fragments thereof, (e.g., Fab′, F(ab′)₂, Fab, Fv and rIgG). See also, Pierce Catalog and Handbook, 1994-1995 (Pierce Chemical Co., Rockford, Ill.). See also, e.g. Kuby, J., Immunology, 3rd Ed., W.H. Freeman & Co., New York (1998). Such non-naturally occurring antibodies can be constructed using solid phase peptide synthesis, can be produced recombinantly or can be obtained, for example, by screening combinatorial libraries consisting of variable heavy chains and variable light chains as described by Huse et al., Science 246:1275-81 (1989), which is incorporated herein by reference. These and other methods of making, for example, chimeric, humanized, CDR-grafted, single chain, and bifunctional antibodies are well known to those skilled in the art (Winter and Harris, Immunol. Today 14:243-6 (1993); Ward et al., Nature 341:544-6 (1989); Harlow and Lane, Antibodies, 511-52, Cold Spring Harbor Laboratory publications, New York, 1988; Hilyard et al., Protein Engineering: A practical approach (IRL Press 1992); Borrebaeck, Antibody Engineering, 2d ed. (Oxford University Press 1995); each of which is incorporated herein by reference).

The term “antibody” includes both polyclonal and monoclonal antibodies. The term also includes genetically engineered forms such as chimeric antibodies (e.g., humanized murine antibodies) and heteroconjugate antibodies (e.g., bispecific antibodies). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Holliger et al. (1993) Proc Natl Acad Sci USA. 90:6444, Gruber et al. (1994) J Immunol:5368, Zhu et al. (1997) Protein Sci 6:781, Hu et al. (1997) Cancer Res. 56:3055, Adams et al. (1993) Cancer Res. 53:4026, and McCartney, et al. (1995) Protein Eng. 8:301.

Typically, an antibody has a heavy and light chain. Each heavy and light chain contains a constant region and a variable region, (the regions are also known as “domains”). Light and heavy chain variable regions contain four “framework” regions interrupted by three hyper-variable regions, also called “complementarity-determining regions” or “CDRs”. The extent of the framework regions and CDRs have been defined. The sequences of the framework regions of different light or heavy chains are relatively conserved within a species. The framework region of an antibody, that is the combined framework regions of the constituent light and heavy chains, serves to position and align the CDRs in three dimensional spaces.

The CDRs are primarily responsible for binding to an epitope of an antigen. The

CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found. References to “VH” refer to the variable region of an immunoglobulin heavy chain of an antibody, including the heavy chain of an Fv, scFv, or Fab. References to “VL” refer to the variable region of an immunoglobulin light chain, including the light chain of an Fv, scFv, dsFv or Fab.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site.

A “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric to antibody, e.g., an enzyme, toxin, hormone, growth factor, drug, etc.; or (b) the variable region, or a portion thereof, is altered, replaced or exchanged with a variable region having a different or altered antigen specificity.

A “humanized antibody” is an immunoglobulin molecule that contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-5 (1986); Riechmann et al., Nature 332:323-7 (1988); and Presta, Curr. Op. Struct. Biol. 2:593-6 (1992)). Humanization can be essentially performed following the method of Winter and co-workers (Jones et al., Nature 321:522-5 (1986); Riechmann et al., Nature 332:323-7 (1988); Verhoeyen et al., Science 239:1534-6 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

The terms “epitope”, “antigenic” and “determinant” refer to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, X-ray crystallography and 2-dimensional nuclear magnetic resonance. See, e.g., Epitope Mapping Protocols in Methods in Molecular Biology, Vol. 66, Glenn E. Morris, Ed (1996).

The terms “non-antibody binding protein” or “non-antibody ligand” or “antigen binding protein” interchangeably refer to antibody mimics that use non-immunoglobulin protein scaffolds, including adnectins, avimers, single chain polypeptide binding molecules, and antibody-like binding peptidomimetics, as discussed in more detail below.

Other compounds have been developed that target and bind to targets in a manner similar to antibodies. Certain of these “antibody mimics” use non-immunoglobulin protein scaffolds as alternative protein frameworks for the variable regions of antibodies.

For example, Ladner et al. (U.S. Pat. No. 5,260,203) describe single polypeptide chain binding molecules with binding specificity similar to that of the aggregated, but molecularly separate, light and heavy chain variable region of antibodies. The single-chain binding molecule contains the antigen binding sites of both the heavy and light variable regions of an antibody connected by a peptide linker and will fold into a structure similar to that of the two peptide antibody. The single-chain binding molecule displays several advantages over conventional antibodies, including, smaller size, greater stability and are more easily modified.

Ku et al. (Proc. Natl. Acad. Sci. USA 92(14):6552-6556 (1995)) discloses an alternative to antibodies based on cytochrome b562. Ku et al. (1995) generated a library in which two of the loops of cytochrome b562 were randomized and selected for binding against bovine serum albumin. The individual mutants were found to bind selectively with BSA similarly with anti-BSA antibodies.

Lipovsek et al. (U.S. Pat. Nos. 6,818,418 and 7,115,396) discloses an antibody mimic featuring a fibronectin or fibronectin-like protein scaffold and at least one variable loop. Known as Adnectins, these fibronectin-based antibody mimics exhibit many of the same characteristics of natural or engineered antibodies, including high affinity and specificity for any targeted ligand. Any technique for evolving new or improved binding proteins can be used with these antibody mimics.

The structure of these fibronectin-based antibody mimics is similar to the structure of the variable region of the IgG heavy chain. Therefore, these mimics display antigen binding properties similar in nature and affinity to those of native antibodies. Further, these fibronectin-based antibody mimics exhibit certain benefits over antibodies and antibody fragments. For example, these antibody mimics do not rely on disulfide bonds for native fold stability, and are, therefore, stable under conditions which would normally break down antibodies. In addition, since the structure of these fibronectin-based antibody mimics is similar to that of the IgG heavy chain, the process for loop randomization and shuffling can be employed in vitro that is similar to the process of affinity maturation of antibodies in vivo.

Beste et al. (Proc. Natl. Acad. Sci. USA 96(5):1898-1903 (1999)) discloses an antibody mimic based on a lipocalin scaffold (Anticalin®). Lipocalins are composed of a β-barrel with four hypervariable loops at the terminus of the protein. Beste (1999), subjected the loops to random mutagenesis and selected for binding with, for example, fluorescein. Three variants exhibited specific binding with fluorescein, with one variant showing binding similar to that of an anti-fluorescein antibody. Further analysis revealed that all of the randomized positions are variable, indicating that Anticalin® would be suitable to be used as an alternative to antibodies.

Anticalins® are small, single chain peptides, typically between 160 and 180 residues, which provides several advantages over antibodies, including decreased cost of production, increased stability in storage and decreased immunological reaction.

Hamilton et al. (U.S. Pat. No. 5,770,380) discloses a synthetic antibody mimic using the rigid, non-peptide organic scaffold of calixarene, attached with multiple variable peptide loops used as binding sites. The peptide loops all project from the same side geometrically from the calixarene, with respect to each other. Because of this geometric confirmation, all of the loops are available for binding, increasing the binding affinity to a ligand. However, in comparison to other antibody mimics, the calixarene-based antibody mimic does not consist exclusively of a peptide, and therefore it is less vulnerable to attack by protease enzymes. Neither does the scaffold consist purely of a peptide, DNA or RNA, meaning this antibody mimic is relatively stable in extreme environmental conditions and has a long life span. Further, since the calixarene-based antibody mimic is relatively small, it is less likely to produce an immunogenic response.

Murali et al. (Cell. Mol. Biol. 49(2):209-216 (2003)) discusses a methodology for reducing antibodies into smaller peptidomimetics, they term “antibody like binding peptidomemetics” (ABiP) which can also be useful as an alternative to antibodies.

Silverman et al. (Nat. Biotechnol. (2005), 23: 1556-1561) discloses fusion proteins that are single-chain polypeptides comprising multiple domains termed “avimers”. Developed from human extracellular receptor domains by in vitro exon shuffling and phage display the avimers are a class of binding proteins somewhat similar to antibodies in their affinities and specificities for various target molecules. The resulting multidomain proteins can comprise multiple independent binding domains that can exhibit improved affinity (in some cases sub-nanomolar) and specificity compared with single-epitope binding proteins. Additional details concerning methods of construction and use of avimers are disclosed, for example, in U.S. Patent App. Pub. Nos. 20040175756, 20050048512, 20050053973, 20050089932 and 20050221384.

In addition to non-immunoglobulin protein frameworks, antibody properties have also been mimicked in compounds comprising RNA molecules and unnatural oligomers (e.g., protease inhibitors, benzodiazepines, purine derivatives and beta-turn mimics) all of which are suitable for use with the present invention.

### III. Diagnosing Cancer:

### III-1. Method for Diagnosing Cancer or a Predisposition for Developing Cancer

The expression of the IMP-1 gene was found to be specifically elevated in patients with cancer. Therefore, the gene identified herein, as well as its transcription and translation products, finds diagnostic utility as a marker for cancer. Accordingly, by measuring the expression of the IMP-1 gene in a cell sample, cancer can be diagnosed. Specifically, the present invention provides a method for diagnosing cancer or a predisposition for developing cancer in a subject by determining the expression level of the IMP-1 gene in the subject.

Cancers that can be diagnosed by the present method include, but are not limited to, lung cancers. The present method is particularly suited for diagnosing NSCLCs.

According to another aspect of the present invention, the predisposition for developing at least any one of such cancers.

In the context of the present invention, the term “diagnosing” is intended to encompass predictions and likelihood analysis. The present method is intended to be used clinically in making decisions concerning treatment modalities, including therapeutic intervention, diagnostic criteria such as disease stages, and disease monitoring and surveillance for cancer. According to the present invention, an intermediate result for examining the condition of a subject may be provided. Such intermediate result may be combined with additional information to assist a doctor, nurse, or other practitioner to diagnose that a subject suffers from the disease. Alternatively, the present invention may be used to detect cancerous cells in a subject-derived tissue, and provide a doctor with useful information to diagnose that the subject suffers from the disease.

A subject to be diagnosed by the present method is preferably a mammal. Exemplary mammals include, but are not limited to, e.g., human, non-human primate, mouse, rat, dog, cat, horse, and cow.

It is preferred to collect a biological sample from the subject to be diagnosed to perform the diagnosis. Any biological material can be used as the biological sample for the determination so long as it includes the objective transcription or translation product of the IMP-1 gene. The biological samples include, but are not limited to, bodily tissues and fluids, such as blood, sputum, and urine. Preferably, the biological sample contains a cell population including an epithelial cell, more preferably a cancerous epithelial cell or an epithelial cell derived from tissue suspected to be cancerous. Further, if necessary, the cell may be purified from the obtained bodily tissues and fluids, and then used as the biological sample.

According to the present invention, the expression level of the IMP-1 gene is determined in the subject-derived biological sample. The expression level can be determined at the transcription (nucleic acid) product level, using methods known in the art. For example, the mRNA of the IMP-1 gene may be quantified using probes by hybridization methods (e.g., Northern hybridization). The detection may be carried out on a chip or an array. The use of an array is preferable for detecting the expression level of a plurality of genes (e.g., various cancer specific genes) including the present IMP-1 gene. Those skilled in the art can prepare such probes utilizing the sequence information of the IMP-1 gene (SEQ ID NO: 11; GenBank Accession No. NM_{—}006546). For example, the cDNA of the IMP-1 gene may be used as the probes. If necessary, the probe may be labeled with a suitable label, such as dyes and isotopes, and the expression level of the gene may be detected as the intensity of the hybridized labels.

Furthermore, the transcription product of the IMP-1 gene may be quantified using primers by amplification-based detection methods (e.g., RT-PCR). Such primers can also be prepared based on the available sequence information of the gene. For example, the primers (SEQ ID NOs: 1 and 2) used in the Example may be employed for the detection by RT-PCR, but the present invention is not restricted thereto.

Specifically, a probe or primer used for the present method hybridizes under stringent, moderately stringent, or low stringency conditions to the mRNA of the IMP-1 gene. As used herein, the phrase “stringent (hybridization) conditions” refers to conditions under which a probe or primer will hybridize to its target sequence, but to no other sequences. Stringent conditions are sequence-dependent and will be different under different circumstances. Specific hybridization of longer sequences is observed at higher temperatures than shorter sequences. Generally, the temperature of a stringent condition is selected to be about 5° C. lower than the thermal melting point (Tm) for a specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes or primers (e.g., 10 to 50 nucleotides) and at least about 60° C. for longer probes or primers. Stringent conditions may also be achieved with the addition of destabilizing agents, such as formamide.

Alternatively, the translation product may be detected for the diagnosis of the present invention. For example, the quantity of the IMP-1 protein may be determined. Illustrative methods for determining the quantity of the protein as the translation product include immunoassay methods that use an antibody specifically recognizing the protein. For example, the antibody specifically recognizing the protein can be prepared by using a polypeptide set forth in SEQ ID NO: 5 or SEQ ID NO: 6 (see the item of ‘D. Preparation of anti-IMP-1 polyclonal antibody’ in EXAMPLE). The antibody may be monoclonal or polyclonal. Furthermore, any fragment or modification (e.g., chimeric antibody, scFv, Fab, F(ab′)₂, Fv, etc.) of the antibody may be used for the detection, so long as the fragment retains the binding ability to the IMP-1 protein. Methods to prepare these kinds of antibodies for the detection of proteins are well known in the art, and any method may be employed in the present invention to prepare such antibodies and equivalents thereof.

As another method to detect the expression level of the IMP-1 gene based on its translation product, the intensity of staining may be observed via immunohistochemical analysis using an antibody against the IMP-1 protein. Namely, the observation of strong staining indicates increased presence of the protein and at the same time high expression level of the IMP-1 gene. NSCLC tissue can be preferably used as a test material for immunohistochemical analysis.

Moreover, in addition to the expression level of the IMP-1 gene, the expression level of other cancer-associated genes, for example, genes known to be differentially expressed in NSCLCs, may also be determined to improve the accuracy of the diagnosis.

The expression level of cancer marker gene including the IMP-1 gene in a biological sample can be considered to be increased if it increases from the control level of the corresponding cancer marker gene by, for example, 10%, 25%, or 50%; or increases to more than 1.1 fold, more than 1.5 fold, more than 2.0 fold, more than 5.0 fold, more than 10.0 fold, or more.

The control level may be determined at the same time with the test biological sample by using a sample(s) previously collected and stored from a subject/subjects whose disease state (cancerous or non-cancerous) is/are known. Alternatively, the control level may be determined by a statistical method based on the results obtained by analyzing previously determined expression level(s) of the IMP-1 gene in samples from subjects whose disease state are known. Furthermore, the control level can be a database of expression patterns from previously tested cells. Moreover, according to an aspect of the present invention, the expression level of the IMP-1 gene in a biological sample may be compared to multiple control levels, which control levels are determined from multiple reference samples. It is preferred to use a control level determined from a reference sample derived from a tissue type similar to that of the patient-derived biological sample. Moreover, it is preferred, to use the standard value of the expression levels of the IMP-1 gene in a population with a known disease state. The standard value may be obtained by any method known in the art. For example, a range of mean±2 S.D. or mean±3 S.D. may be used as standard value.

In the context of the present invention, a control level determined from a biological sample that is known not to be cancerous is called “normal control level”. On the other hand, if the control level is determined from a cancerous biological sample, it will be called “cancerous control level”.

When the expression level of the IMP-1 gene is increased compared to the normal control level or is similar to the cancerous control level, the subject may be diagnosed to be suffering from or at a risk of developing cancer. Furthermore, a similarity in the gene expression pattern between the sample and the reference which is cancerous indicates that the subject is suffering from or at a risk of developing cancer.

The difference between the expression levels of a test biological sample and the control level can be normalized to the expression level of control nucleic acids, e.g. housekeeping genes whose expression levels are known not to differ depending on the cancerous or non-cancerous state of the cell. Exemplary control genes include, but are not limited to, β-actin, glyceraldehyde 3-phosphate dehydrogenase, and ribosomal protein P1.

### III-2. Assessing Efficacy of Cancer Treatment

The IMP-1 gene differentially expressed between normal and cancerous cells also allow for the course of cancer treatment to be monitored, and the above-described method for diagnosing cancer can be applied for assessing the efficacy of a treatment on cancer. Specifically, the efficacy of a treatment on cancer can be assessed by determining the expression level of the IMP-1 gene in a cell(s) derived from a subject undergoing the treatment. If desired, test cell populations are obtained from the subject at various time points, before, during, and/or after the treatment. The expression level of the IMP-1 gene can be, for example, determined following the method described above under the item of ‘I-1. Method for diagnosing cancer or a predisposition for developing cancer’. In the context of the present invention, it is preferred that the control level to which the detected expression level is compared is determined from the IMP-1 gene expression in a cell(s) not exposed to the treatment of interest.

If the expression level of the IMP-1 gene is compared to a control level that is determined from a normal cell or a cell population containing no cancer cell, a similarity in the expression level indicates that the treatment of interest is efficacious and a difference in the expression level indicates less favorable clinical outcome or prognosis of that treatment. On the other hand, if the comparison is conducted against a control level that is determined from a cancer cell or a cell population containing a cancer cell(s), a difference in the expression level indicates efficacious treatment, while a similarity in the expression level indicates less favorable clinical outcome or prognosis.

Furthermore, the expression levels of the IMP-1 gene before and after a treatment can be compared according to the present method to assess the efficacy of the treatment. Specifically, the expression level detected in a subject-derived biological sample after a treatment (i.e., post-treatment level) is compared to the expression level detected in a biological sample obtained prior to treatment onset from the same subject (i.e., pre-treatment level). A decrease in the post-treatment level compared to the pre-treatment level indicates that the treatment of interest is efficacious while an increase in or similarity of the post-treatment level to the pre-treatment level indicates less favorable clinical outcome or prognosis.

As used herein, the term “efficacious” indicates that the treatment leads to a reduction in the expression of a pathologically up-regulated gene, an increase in the expression of a pathologically down-regulated gene or a decrease in size, prevalence, or metastatic potential of carcinoma in a subject. When a treatment of interest is applied prophylactically, “efficacious” means that the treatment retards or prevents the forming of tumor or retards, prevents, or alleviates at least one clinical symptom of cancer. Assessment of the state of tumor in a subject can be made using standard clinical protocols.

In addition, efficaciousness of a treatment can be determined in association with any known method for diagnosing cancer. Cancers can be diagnosed, for example, by identifying symptomatic anomalies, e.g., weight loss, abdominal pain, back pain, anorexia, nausea, vomiting and generalized malaise, weakness, and jaundice.

III-3. Assessing Prognosis of Subject with Cancer

The method for diagnosing cancer described above can also be used for assessing or determining the prognosis of cancer in a subject. Thus, the present invention also provides a method for assessing or determining the prognosis of a subject with cancer. The expression level of the IMP-1 gene can be, for example, determined following the method described above under the item of ‘I-1. Method for diagnosing cancer or a predisposition for developing cancer’. For example, the expression level of the IMP-1 gene in biological samples derived from patients over a spectrum of disease stages can be used as control levels to be compared with the expression level of the gene detected for a subject. By comparing the expression level of the IMP-1 gene in a subject and the control level(s) the prognosis of the subject can be assessed. Alternatively, by comparing over time the pattern of expression levels in a subject, the prognosis of the subject can be assessed.

For example, an increase in the expression level of IMP-1 gene in a subject as compared to a normal control level indicates less favorable prognosis. Conversely, a similarity in the expression level as compared to normal control level indicates a more favorable prognosis for the subject.

According to the present invention, an intermediate result may be provided in addition to other test results for assessing the prognosis of a subject. Such intermediate result may assist a doctor, nurse, or other practitioner to assess, determine, or estimate the prognosis of a subject. Additional information that may be considered, in combination with the intermediate result obtained by the present invention, to assess prognosis includes clinical symptoms and physical conditions of a subject.

### IV. Screening Methods:

Using the IMP-1 gene, polypeptides encoded by the gene or fragments thereof, or transcriptional regulatory region of the gene, it is possible to screen for agents that alter the expression of the gene or the biological activity of a polypeptide encoded by the gene. Such agents can be used as pharmaceuticals for treating or preventing cancer. Thus, the present invention provides methods of identifying agents for treating or preventing cancer using the IMP-1 gene, polypeptide encoded by the gene or fragments thereof, or transcriptional regulatory region of the gene.

An agent identified by the screening method of the present invention is an agent that is expected to inhibit the expression of the IMP-1 gene or the activity of the translation product of the gene, and thus, is a candidate for treating or preventing diseases attributed to, for example, cell proliferative diseases, such as cancer. The agents are expected to treat or prevent cancer selected from the group of NSCLCs. Namely, the agents identified through the present methods are expected to have a clinical benefit and can be further tested for an ability to prevent cancer cell growth in animal models or test subjects.

In the context of the present invention, agents to be identified through the present screening methods may be any compound or composition, including several compounds. Furthermore, the test agent exposed to a cell or protein according to the screening methods of the present invention may be a single compound or a combination of compounds. When a combination of compounds is used in the methods, the compounds may be contacted sequentially or simultaneously.

Any test agent, for example, cell extracts, cell culture supernatant, products of fermenting microorganism, extracts from marine organism, plant extracts, purified or crude proteins, peptides, non-peptide compounds, synthetic micromolecular compounds (including nucleic acid constructs, such as antisense RNA, siRNA, ribozymes, etc.) and natural compounds can be used in the screening methods of the present invention. The test agent of the present invention can be also obtained using any of the numerous approaches in combinatorial library methods known in the art, including, but not limited to, (1) biological libraries, (2) spatially addressable parallel solid phase or solution phase libraries, (3) synthetic library methods requiring deconvolution, (4) the “one-bead one-compound” library method and (5) synthetic library methods using affinity chromatography selection. The biological library methods using affinity chromatography selection is limited to peptide libraries, while the other four approaches are applicable to peptide, non-peptide oligomer or small molecule libraries of compounds (Lam, Anticancer Drug Des 1997, 12: 145-67). Examples of methods for the synthesis of molecular libraries can be found in the art (DeWitt et al., Proc Natl Acad Sci USA 1993, 90: 6909-13; Erb et al., Proc Natl Acad Sci USA 1994, 91: 11422-6; Zuckermann et al., J Med Chem 37: 2678-85, 1994; Cho et al., Science 1993, 261: 1303-5; Carell et al., Angew Chem Int Ed Engl 1994, 33: 2059; Carell et al., Angew Chem Int Ed Engl 1994, 33: 2061; Gallop et al., J Med Chem 1994, 37: 1233-51). Libraries of compounds may be presented in solution (see Houghten, Bio/Techniques 1992, 13: 412-21) or on beads (Lam, Nature 1991, 354: 82-4), chips (Fodor, Nature 1993, 364: 555-6), bacteria (U.S. Pat. No. 5,223,409), spores (U.S. Pat. Nos. 5,571,698; 5,403,484, and 5,223,409), plasmids (Cull et al., Proc Natl Acad Sci USA 1992, 89: 1865-9) or phage (Scott and Smith, Science 1990, 249: 386-90; Devlin, Science 1990, 249: 404-6; Cwirla et al., Proc Natl Acad Sci USA 1990, 87: 6378-82; Felici, J Mol Biol 1991, 222: 301-10; US Pat. Application 2002103360).

A compound in which a part of the structure of the compound identified by any of the present screening methods is converted by addition, deletion and/or replacement, is included in the agents obtained by the screening methods of the present invention.

Furthermore, when the screened test agent is a protein, or a DNA encoding the protein, either the whole amino acid sequence of the protein may be determined to deduce the nucleic acid sequence coding for the protein, or partial amino acid sequence of the obtained protein may be analyzed to prepare an oligo DNA as a probe based on the sequence, and screen cDNA libraries with the probe to obtain a DNA encoding the protein. The obtained DNA find use in preparing the test agent which is a candidate for treating or preventing cancer.

### IV-1. Protein Based Screening Methods

According to the present invention, the expression of the IMP-1 gene was suggested to be crucial for the growth and/or survival of cancer cells. Therefore, it was considered that agents which suppress the function of the polypeptide encoded by the gene inhibit the growth and/or survival of cancer cells, and find use in treating or preventing cancer. Thus, the present invention provides methods of identifying an agent for treating or preventing cancer, using the IMP-1 polypeptide.

In addition to the IMP-1 polypeptide, fragments of the polypeptide may be used in the context of the present screening methods, so long as at least one biological activity of natural occurring IMP-1 polypeptide is retained.

The polypeptide or fragments thereof may be further linked to other substances so long as the resulting polypeptide and fragments retain at least one biological activity of the originating peptide. Usable substances include: peptides, lipids, sugar and sugar chains, acetyl groups, natural and synthetic polymers, etc. These kinds of modifications may be performed to confer additional functions or to stabilize the polypeptide and fragments.

The polypeptide or fragments used for the present method may be obtained from nature as naturally occurring proteins via conventional purification methods or through chemical synthesis based on the selected amino acid sequence. For example, conventional peptide synthesis methods that can be adopted for the synthesis includes:
1) Peptide Synthesis, Interscience, New York, 1966;
2) The Proteins, Vol. 2, Academic Press, New York, 1976;
3) Peptide Synthesis (in Japanese), Maruzen Co., 1975;
4) Basics and Experiment of Peptide Synthesis (in Japanese), Maruzen Co., 1985;
5) Development of Pharmaceuticals (second volume) (in Japanese), Vol. 14 (peptide synthesis), Hirokawa, 1991;
6) WO99/67288; and
7) Barany G. & Merrifield R. B., Peptides Vol. 2, “Solid Phase Peptide Synthesis”, Academic Press, New York, 1980, 100-118.

Alternatively, the protein may be obtained adopting any known genetic engineering methods for producing polypeptides (e.g., Morrison J., J Bacteriology 1977, 132: 349-51; Clark-Curtiss & Curtiss, Methods in Enzymology (eds. Wu et al.) 1983, 101: 347-62). For example, first, a suitable vector including a polynucleotide encoding the objective protein in an expressible form (e.g., downstream of a regulatory sequence including a promoter) is prepared, transformed into a suitable host cell, and then the host cell is cultured to produce the protein. More specifically, a gene encoding the IMP-1 polypeptide is expressed in host (e.g., animal) cells and such by inserting the gene into a vector for expressing foreign genes, such as pSV2neo, pcDNA I, pcDNA3.1, pCAGGS, or pCD8. A promoter may be used for the expression. Any commonly used promoters may be employed including, for example, the SV40 early promoter (Rigby in Williamson (ed.), Genetic engineering, vol. 3. Academic Press, London, 1982, 83-141), the EF-α promoter (Kim et al., Gene 1990, 91:217-23), the CAG promoter (Niwa et al., Gene 1991, 108:193), the RSV LTR promoter (Cullen, Methods in Enzymology 1987, 152:684-704), the SRα promoter (Takebe et al., Mol Cell Biol 1988, 8:466), the CMV immediate early promoter (Seed et al., Proc Natl Acad Sci USA 1987, 84:3365-9), the SV40 late promoter (Gheysen et al., J Mol Appl Genet 1982, 1:385-94), the Adenovirus late promoter (Kaufman et al., Mol Cell Biol 1989, 9:946), the HSV TK promoter, and such. The introduction of the vector into host cells to express the IMP-1 gene can be performed according to any methods, for example, the electroporation method (Chu et al., Nucleic Acids Res 1987, 15:1311-26), the calcium phosphate method (Chen et al., Mol Cell Biol 1987, 7:2745-52), the DEAE dextran method (Lopata et al., Nucleic Acids Res 1984, 12:5707-17; Sussman et al., Mol Cell Biol 1984, 4:1641-3), the Lipofectin method (Derijard B, Cell 1994, 7:1025-37); Lamb et al., Nature Genetics 1993, 5:22-30; Rabindran et al., Science 1993, 259:230-4), and such.

The IMP-1 protein may also be produced in vitro adopting an in vitro translation system.

The IMP-1 polypeptide to be contacted with a test agent can be, for example, a purified polypeptide, a soluble protein, or a fusion protein fused with other polypeptides.

IV-1-1. Identifying Agents that Bind to IMP-1 Polypeptide

An agent that binds to a protein is likely to alter the expression of the gene coding for the protein or the biological activity of the protein. Thus, in one aspect, the present invention provides a method of screening for an agent for treating or preventing cancer, which includes the steps of:

a) contacting a test agent with the IMP-1 polypeptide or a functional fragment thereof,

b) detecting the binding between the polypeptide (or fragment) and the test agent; and

c) selecting the test agent that binds to the polypeptide (or fragment).

The binding of a test agent to the IMP-1 polypeptide may be, for example, detected by immunoprecipitation using an antibody against the polypeptide. Therefore, for the purpose for such detection, it is preferred that the IMP-1 polypeptide or functional fragments thereof used for the screening contains an antibody recognition site. The antibody used for the screening may be one that recognizes an antigenic region (e.g., epitope) of the present IMP-1 polypeptide which preparation methods are well known in the art, and any method may be employed in the present invention to prepare such antibodies and equivalents thereof.

Alternatively, the IMP-1 polypeptide or a functional fragment thereof may be expressed as a fusion protein including at its N- or C-terminus a recognition site (epitope) of a monoclonal antibody, whose specificity has been revealed, to the N- or C-terminus of the polypeptide. A commercially available epitope-antibody system can be used (Experimental Medicine 1995, 13:85-90). Vectors which can express a fusion protein with, for example, β-galactosidase, maltose binding protein, glutathione S-transferase, green florescence protein (GFP), and such by the use of its multiple cloning sites are commercially available and can be used for the present invention. Furthermore, fusion proteins containing much smaller epitopes to be detected by immunoprecipitation with an antibody against the epitopes are also known in the art (Experimental Medicine 1995, 13:85-90). Such epitopes consisting of several dozen amino acids so as not to change the property of the IMP-1 polypeptide or fragments thereof can also be used in the present invention. Examples include, but are not limited to, polyhistidine (His-tag), influenza aggregate HA, human c-myc, FLAG, Vesicular stomatitis virus glycoprotein (VSV-GP), T7 gene 10 protein (T7-tag), human simple herpes virus glycoprotein (HSV-tag), E-tag (an epitope on monoclonal phage), and such.

Glutathione S-transferase (GST) is also well-known as the counterpart of the fusion protein to be detected by immunoprecipitation. When GST is used as the protein to be fused with the IMP-1 polypeptide or fragment thereof to form a fusion protein, the fusion protein can be detected either with an antibody against GST or a substance specifically binding to GST, i.e., such as glutathione (e.g., glutathione-Sepharose 4B).

In immunoprecipitation, an immune complex is formed by adding an antibody (recognizing the IMP-1 polypeptide or a functional fragment thereof itself, or an epitope tagged to the polypeptide or fragment) to the reaction mixture of the IMP-1 polypeptide and the test agent. If the test agent has the ability to bind the polypeptide, then the formed immune complex will be composed of the IMP-1 polypeptide, the test agent, and the antibody. On the contrary, if the test agent is devoid of such ability, then the formed immune complex only include the IMP-1 polypeptide and the antibody. Therefore, the binding ability of a test agent to IMP-1 polypeptide can be examined by, for example, measuring the size of the formed immune complex. Any method for detecting the size of a substance can be used, including chromatography, electrophoresis, and such. For example, when mouse IgG antibody is used for the detection, Protein A or Protein G sepharose can be used for quantitating the formed immune complex.

For more details on immunoprecipitation see, for example, Harlow et al., Antibodies, Cold Spring Harbor Laboratory publications, New York, 1988, 511-52.

Furthermore, the IMP-1 polypeptide or a functional fragment thereof used for the screening of agents that bind to thereto may be bound to a carrier. Example of carriers that may be used for binding the polypeptides include insoluble polysaccharides, such as agarose, cellulose and dextran; and synthetic resins, such as polyacrylamide, polystyrene and silicon; preferably commercially available beads and plates (e.g., multi-well plates, biosensor chip, etc.) prepared from the above materials may be used. When using beads, they may be filled into a column. Alternatively, the use of magnetic beads is also known in the art, and enables to readily isolate polypeptides and agents bound on the beads via magnetism.

The binding of a polypeptide to a carrier may be conducted according to routine methods, such as chemical bonding and physical adsorption. Alternatively, a polypeptide may be bound to a carrier via antibodies specifically recognizing the protein. Moreover, binding of a polypeptide to a carrier can also be conducted by means of interacting molecules, such as the combination of avidin and biotin.

Screening methods using such carrier-bound IMP-1 polypeptide or functional fragments thereof include, for example, the steps of contacting a test agent to the carrier-bound polypeptide, incubating the mixture, washing the carrier, and detecting and/or measuring the agent bound to the carrier. The binding may be carried out in buffer, for example, but are not limited to, phosphate buffer and Tris buffer, as long as the buffer does not inhibit the binding.

An exemplary screening method wherein such carrier-bound IMP-1 polypeptide or fragments thereof and a composition (e.g., cell extracts, cell lysates, etc.) are used as the test agent includes affinity chromatography. For example, the IMP-1 polypeptide may be immobilized on a carrier of an affinity column, and a test agent, containing a substance capable of binding to the polypeptides, is applied to the column. After loading the test agent, the column is washed, and then the substance bound to the polypeptide is eluted with an appropriate buffer.

A biosensor using the surface plasmon resonance phenomenon may be used as a mean for detecting or quantifying the bound agent in the present invention. When such a biosensor is used, the interaction between the IMP-1 polypeptide and a test agent can be observed real-time as a surface plasmon resonance signal, using only a minute amount of the polypeptide and without labeling (for example, BIAcore, Pharmacia). Therefore, it is possible to evaluate the binding between the polypeptide and test agent using a biosensor such as BIAcore.

Methods of screening for molecules that bind to a specific protein among synthetic chemical compounds, or molecules in natural substance banks or a random phage peptide display library by exposing the specific protein immobilized on a carrier to the molecules, and methods of high-throughput screening based on combinatorial chemistry techniques (Wrighton et al., Science 1996, 273:458-64; Verdine, Nature 1996, 384:11-3) to isolate not only proteins but chemical compounds are also well-known to those skilled in the art. These methods can also be used for screening agents (including agonist and antagonist) that bind to the IMP-1 protein or fragments thereof.

When the test agent is a protein, for example, West-Western blotting analysis (Skolnik et al., Cell 1991, 65:83-90) can be used for the present method. Specifically, a protein binding to the IMP-1 polypeptide can be obtained by preparing first a cDNA library is prepared from cells, tissues, organs, or cultured cells (e.g., NSCLC) expected to express at least one protein binding to the IMP-1 polypeptide using a phage vector (e.g., ZAP), expressing the proteins encoded by the vectors of the cDNA library on LB-agarose, fixing the expressed proteins on a filter, reacting the purified and labeled IMP-1 polypeptide with the above filter, and detecting the plaques expressing proteins to which the IMP-1 polypeptide has bound according to the label of the IMP-1 polypeptide. Labeling substances such as radioisotope (e.g., 3H, 14C, ³²P, ³³P, ³⁵S, ¹²⁵I, ¹³¹I), enzymes (e.g., alkaline phosphatase, horseradish peroxidase, β-galactosidase, β-glucosidase), fluorescent substances (e.g., fluorescein isothiosyanete (FITC), rhodamine) and biotin/avidin, may be used for the labeling of IMP-1 polypeptide in the present method. When the protein is labeled with radioisotope, the detection or measurement can be carried out by liquid scintillation. Alternatively, when the protein is labeled with an enzyme, it can be detected or measured by adding a substrate of the enzyme to detect the enzymatic change of the substrate, such as generation of color, with absorptiometer. Further, in case where a fluorescent substance is used as the label, the bound protein may be detected or measured using fluorophotometer.

Moreover, the IMP-1 polypeptide bound to the protein can be detected or measured by utilizing an antibody that specifically binds to the IMP-1 polypeptide, or a peptide or polypeptide (for example, GST) that is fused to the IMP-1 polypeptide. In case of using an antibody in the present screening, the antibody is preferably labeled with one of the labeling substances mentioned above, and detected or measured based on the labeling substance. Alternatively, the antibody against the IMP-1 polypeptide may be used as a primary antibody to be detected with a secondary antibody that is labeled with a labeling substance. Furthermore, the antibody bound to the IMP-1 polypeptide in the present screening may be detected or measured using protein G or protein A column.

Alternatively, in another embodiment of the screening method of the present invention, two-hybrid system utilizing cells may be used (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton et al., Cell 1992, 68:597-612” and “Fields et al., Trends Genet 1994, 10:286-92”). In two-hybrid system, IMP-1 polypeptide or a fragment thereof is fused to the SRF-binding region or GAL4-binding region and expressed in yeast cells. A cDNA library is prepared from cells expected to express at least one protein binding to the IMP-1 polypeptide, such that the library, when expressed, is fused to the VP16 or GAL4 transcriptional activation region. The cDNA library is then introduced into the above yeast cells and the cDNA derived from the library is isolated from the positive clones detected (when a protein binding to the IMP-1 polypeptide is expressed in the yeast cells, the binding of the two activates a reporter gene, making positive clones detectable). A protein encoded by the cDNA can be prepared by introducing the cDNA isolated above to E. coli and expressing the protein.

As a reporter gene, for example, Ade2 gene, lacZ gene, CAT gene, luciferase gene and such can be used in addition to the HIS3 gene.

The agent identified by this screening is a candidate for agonists or antagonists of the IMP-1 polypeptide. The term “agonist” refers to molecules that activate the function of the polypeptide by binding thereto. On the other hand, the term “antagonist” refers to molecules that inhibit the function of the polypeptide by binding thereto. Moreover, an agent isolated by this screening as an antagonist is a candidate that inhibits the in vivo interaction of the IMP-1 polypeptide with molecules (including nucleic acids (RNAs and DNAs) and proteins).

### IV-1-2. Identifying Agents by Detecting Biological Activity of the Imp-1 Polypeptide

According to the present invention, the expression of the IMP-1 gene was shown to by crucial for the growth and/or survival of cancer cells. Therefore, agents that suppress or inhibit the biological function of the translational product of the IMP-1 gene is considered to serve as candidates for treating or preventing cancer. Thus, the present invention also provides a method of screening for a compound for treating or preventing cancer using the IMP-1 polypeptide or fragments thereof including the steps as follows:

a) contacting a test agent with the IMP-1 polypeptide or a functional fragment thereof, and

b) detecting the biological activity of the polypeptide or fragment of step (a); and

Any polypeptide can be used for the screening so long as it has one biological activity of the IMP-1 polypeptide that can be used as an index in the present screening method. Since the IMP-1 polypeptide has the activity of promoting cell proliferation of cancer cells, biological activities of the IMP-1 polypeptide that can be used as an index for the screening include such cell-proliferating activity of the human IMP-1 polypeptide. For example, a human IMP-1 polypeptide can be used and polypeptides functionally equivalent thereto including functional fragments thereof can also be used. Such polypeptides may be expressed endogenously or exogenously by suitable cells.

When the biological activity to be detected in the present method is cell proliferation, it can be detected, for example, by preparing cells which express the IMP-1 polypeptide or a functional fragment thereof, culturing the cells in the presence of a test agent, and determining the speed of cell proliferation, measuring the cell cycle and such, as well as by detecting wound-healing activity, conducting Matrigel invasion assay and measuring the colony forming activity.

According to an aspect of the present invention, the screening further includes, after the above step (b), the step of

c) selecting the test agent that suppresses the biological activity of the polypeptide as compared to the biological activity detected in the absence of the test agent.

The agent isolated by this screening is a candidate for an antagonist of the IMP-1 polypeptide, and thus, is a candidate that inhibits the in vivo interaction of the polypeptide with molecules (including nucleic acids (RNAs and DNAs) and proteins).

Furthermore, using RNA-immunoprecipitation experiments coupled with cDNA microarrays (IP-microarray), dozens of candidate mRNAs that were likely to be associated with IMP-1 in NSCLC cells were identified (see Table 3). IMP-1 may be required for the transport of certain mRNAs that play essential roles in embryogenesis and carcinogenesis. Therefore, it is postulated that proliferating germ-cells or cancer-cells may actively distribute indispensable mRNAs in cells through the transporting system involved in the IMP-1 protein-mRNA complex. The evidence that IMP-1 associates with various mRNAs encoding proteins involved in cell-cycle progression, cell invasion and migration, and various types of enzymatic activities, supports this premise. Therefore, the biological activity of IMP-1 for an index of screening method can be a mRNA binding ability. Thus, the present invention also provides a method of screening for a compound for treating or preventing cancer using the IMP-1 expression cells including the steps as follows:
a) contacting a test agent with a cell expressing the IMP-1 protein or functional equivalent thereof and mRNA(s) of one or more gene(s) selected from Table.3;
b) detecting the binding of the IMP-1 protein and the mRNA(s); and
c) selecting the test agent that reduces the binding of the IMP-1 protein and the mRNA(s) as compared to that detected in the absence of the test agent.

Furthermore, the present invention provides a method of screening for a compound for treating or preventing cancer using the IMP-1 polypeptide or fragments thereof including the steps as follows:
a) contacting a test agent with a IMP-1 protein or functional equivalent thereof and mRNA(s) of one or more gene(s) selected from Table.3 or functional equivalent thereof
b) detecting the binding of the IMP-1 protein and the mRNA(s); and
c) selecting the test agent that reduces the binding of the IMP-1 protein and the mRNA(s) as compared to that detected in the absence of the test agent.

The IMP-1 protein contains four KH motifs that can bind RNA in vitro and two PRMs (RNA recognition motif) that are found in a variety of RNA binding proteins, and four KH motifs are necessary to binding mRNA (Nielsen F C, et al., J Cell Sci. 2002 May 15; 115(Pt 10):2087-97). Therefore the functional equivalent of IMP-1 for above mentioned screening methods may be a peptide that retains these KH motifs (the position at 194aa-265aa, 275aa-348aa, 404aa-475aa and 486aa-558aa), e.g. a peptide fragment consisting of amino acid sequence of 197aa-577aa of SEQ ID NO: 12 (Nielsen F C, et al., J Cell Sci. 2002 May 15; 115(Pt 10):2087-97). The mRNA binding ability can be detected, for example, by combination of RNA-immunoprecipitation by IMP-1 specific antibodies and subsequently a appropriate gene amplification methods, e.g. RT-PCR, described in the item of ‘I. RNA-immunoprecipitation and cDNA microarray screening for identification of IMP-1-associated mRNAs’ in EXAMPLE. The cell or the cell population used for such identification may be any cell or any population of cells so long as it expresses the IMP-1 gene. For example, the cell or population may be or contain cells expressing the functional IMP-1 protein include, for example, cell lines established from cancers (e.g., A549). Furthermore, the cell or population may be or contain a cell which has been transfected with IMP-1 gene. Alternatively, the mRNA binding ability can be detected by in vitro methods, the IMP-1 polypeptide to functional equivalent thereof and mRNA described in Table.3 or their fragment that can binding to IMP-1 polypeptide are incubated in a appropriate condition to binding each other, then detected the binding by e.g. gel-shift assay.

### IV-2. Nucleotide Based Screening Methods

### IV-2-1. Screening Method Using IMP-1 Gene

As discussed in detail above, by controlling the expression level of the IMP-1 gene, one can control the onset and progression of cancer. Thus, agents that may be used in the treatment or prevention of cancers can be identified through screenings that use the expression levels of IMP-1 gene as indices. In the context of the present invention, such screening may include, for example, the following steps:

a) contacting a test agent with a cell expressing the IMP-1 gene;

b) detecting the expression level of the IMP-1 gene; and

c) selecting the test agent that reduces the expression level of the IMP-1 gene as compared to a level detected in the absence of the test agent.

An agent that inhibits the expression of the IMP-1 gene or the activity of its gene product can be identified by contacting a cell expressing the IMP-1 gene with a test agent and then determining the expression level of the IMP-1 gene. Naturally, the identification may also be performed using a population of cells that express the gene in place of a single cell. A decreased expression level detected in the presence of an agent as compared to the expression level in the absence of the agent indicates the agent as being an inhibitor of the IMP-1 gene, suggesting the possibility that the agent is useful for inhibiting cancer, thus a candidate agent to be used for the treatment or prevention of cancer.

The expression level of a gene can be estimated by methods well known to one skilled in the art. The expression level of the IMP-1 gene can be, for example, determined following the method described above under the item of ‘I-1. Method for diagnosing cancer or a predisposition for developing cancer’.

The cell or the cell population used for such identification may be any cell or any population of cells so long as it expresses the IMP-1 gene. For example, the cell or population may be or contain an epithelial cell derived from a tissue. Alternatively, the cell or population may be or contain an immortalized cell derived from a carcinoma cell, including those derived from NSCLCs. Cells expressing the IMP-1 gene include, for example, cell lines established from cancers (e.g., A549). Furthermore, the cell or population may be or contain a cell which has been transfected with IMP-1 gene.

The present method permits the screening of various agents mentioned above and is particularly suited for identifying functional nucleic acid molecules including antisense RNA, siRNA, and such.

### IV-2-2. Screening Method Using Transcriptional Regulatory Region of IMP-1 Gene

According to another aspect, the present invention provides a method which includes the following steps of:

a) contacting a test agent with a cell into which a vector, composed of the transcriptional regulatory region of the IMP-1 gene and a reporter gene that is expressed under the control of the transcriptional regulatory region, has been introduced;

b) detecting the expression or activity of said reporter gene; and

c) selecting the test agent that reduces the expression or activity of said reporter gene as compared to a level detected in the absence of the test agent.

Suitable reporter genes and host cells are well known in the art. The reporter construct required for the screening can be prepared using the transcriptional regulatory region of the IMP-1 gene, which can be obtained as a nucleotide segment containing the transcriptional regulatory region from a genome library based on the nucleotide sequence information of the gene.

The transcriptional regulatory region may be, for example, the promoter sequence of the IMP-1 gene. The reporter construct required for the screening can be prepared by connecting reporter gene sequence to the transcriptional regulatory region of IMP-1 gene. The transcriptional regulatory region of IMP-1 gene herein is the region from start codon to at least 500 bp upstream, preferably 1000 bp, more preferably 5000 or 10000 bp upstream. A nucleotide segment containing the transcriptional regulatory region can be isolated from a genome library or can be propagated by PCR. Methods for identifying a transcriptional regulatory region, and also assay protocol are well known (Molecular Cloning third edition chapter 17, 2001, Cold Springs Harbor Laboratory Press).

When a cell(s) transfected with a reporter gene that is operably linked to the regulatory sequence (e.g. promoter sequence) of the IMP-1 gene is used, an agent can be identified as inhibiting or enhancing the expression of the IMP-1 gene through detecting the expression level of the reporter gene product.

As a reporter gene, for example, Ade2 gene, lacZ gene, CAT gene, luciferase gene, HIS3 gene, and such well-known in the art can be used. Methods for detection of the expression of these genes are well known in the art.

IV-3. Selecting Therapeutic Agents that are Appropriate for a Particular Individual

Differences in the genetic makeup of individuals can result in differences in their relative abilities to metabolize various drugs. An agent that is metabolized in a subject to act as an anti-tumor agent can manifest itself by inducing a change in a gene expression pattern in the subject's cells from that characteristic of a cancerous state to a gene expression pattern characteristic of a non-cancerous state. Accordingly, the IMP-1 gene differentially expressed between cancerous and non-cancerous cells disclosed herein allow for a putative therapeutic or prophylactic inhibitor of cancer to be tested in a test cell population from a selected subject in order to determine if the agent is a suitable inhibitor of cancer in the subject.

To identify an inhibitor of cancer that is appropriate for a specific subject, a test cell population from the subject is exposed to a candidate therapeutic agent, and the expression of IMP-1 gene is determined.

In the context of the method of the present invention, test cell populations contain cancer cells expressing the IMP-1 gene. Preferably, the test cell is an epithelial cell.

Specifically, a test cell population may be incubated in the presence of a candidate therapeutic agent and the expression of the IMP-1 gene in the test cell population may be measured and compared to one or more reference profiles, e.g., a cancerous reference expression profile or a non-cancerous reference expression profile.

A decrease in the expression of the IMP-1 gene in a test cell population relative to a reference cell population containing cancer indicates that the agent has therapeutic potential.

### V. Pharmaceutical Compositions for Treating or Preventing Cancers:

The agents identified by any of the screening methods of the present invention, antisense nucleic acids and siRNAs of the IMP-1 gene, and antibodies against the IMP-1 polypeptide inhibit or suppress the expression of the IMP-1 gene, or the biological activity of the IMP-1 polypeptide and inhibit or disrupts cell cycle regulation and cell proliferation. Thus, the present invention provides compositions for treating or preventing cancer, which compositions include agents identified by any of the screening methods of the present invention, antisense nucleic acids and siRNAs of the IMP-1 gene, or antibodies against the IMP-1 polypeptide. The present compositions can be used for treating or preventing cancer such as NSCLCs.

The compositions may be used as pharmaceuticals for humans and other mammals, such as mice, rats, guinea-pigs, rabbits, cats, dogs, sheep, pigs, cattle, monkeys, baboons, and chimpanzees.

In the context of the present invention, suitable pharmaceutical formulations for the active ingredients of the present invention detailed below (including screened agents, antisense nucleic acids, siRNA, antibodies, etc.) include those suitable for oral, rectal, nasal, topical (including buccal and sub-lingual), vaginal or parenteral (including intramuscular, subcutaneous and intravenous) administration, or for administration by inhalation or insufflation. Preferably, administration is intravenous. The formulations are optionally packaged in discrete dosage units.

Pharmaceutical formulations suitable for oral administration include capsules, microcapsules, cachets and tablets, each containing a predetermined amount of active ingredient. Suitable formulations also include powders, elixirs, granules, solutions, suspensions and emulsions. The active ingredient is optionally administered as a bolus electuary or paste. Alternatively, according to needs, the pharmaceutical composition may be administered non-orally, in the form of injections of sterile solutions or suspensions with water or any other pharmaceutically acceptable liquid. For example, the active ingredients of the present invention can be mixed with pharmaceutically acceptable carriers or media, specifically, sterilized water, physiological saline, plant-oils, emulsifiers, suspending agents, surfactants, stabilizers, flavoring agents, excipients, vehicles, preservatives, binders, and such, in a unit dose form required for generally accepted drug implementation. The amount of active ingredient contained in such a preparation makes a suitable dosage within the indicated range acquirable.

Examples of additives that can be admixed into tablets and capsules include, but are not limited to, binders, such as gelatin, corn starch, tragacanth gum and arabic gum; excipients, such as crystalline cellulose; swelling agents, such as corn starch, gelatin and alginic acid; lubricants, such as magnesium stearate; sweeteners, such as sucrose, lactose or saccharin; and flavoring agents, such as peppermint, Gaultheria adenothrix oil and cherry. A tablet may be made by compression or molding, optionally with one or more formulational ingredients. Compressed tablets may be prepared by compressing in a suitable machine the active ingredients in a free-flowing form such as powder or granules, optionally mixed with a binder, lubricant, inert diluent, lubricating, surface active or dispersing agent. Molded tablets may be made via molding in a suitable machine a mixture of the powdered compound moistened with an inert liquid diluent. The tablets may be coated according to methods well known in the art. The tablets may optionally be formulated so as to provide slow or controlled release of the active ingredient in vivo. A package of tablets may contain one tablet to be taken on each of the month.

Furthermore, when the unit-dosage form is a capsule, a liquid carrier, such as oil, can be further included in addition to the above ingredients.

Oral fluid preparations may be in the form of, for example, aqueous or oily suspensions, solutions, emulsions, syrups or elixirs, or may be presented as a dry product for reconstitution with water or other suitable vehicle prior to use. Such liquid preparations may contain conventional additives such as suspending agents, emulsifying agents, non-aqueous vehicles (which may include edible oils) or preservatives.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit dose or multi-dose containers, for example sealed ampoules and vials, and may be stored in a freeze-dried (lyophilized) condition requiring only the addition of the sterile liquid carrier, for example, saline, water-for-injection, immediately prior to use. Alternatively, the formulations may be presented for continuous infusion. Extemporaneous injection solutions and suspensions may be prepared from sterile powders, granules and tablets of the kind previously described.

Moreover, sterile composites for injection can be formulated following normal drug implementations using vehicles, such as distilled water, suitable for injection. Physiological saline, glucose, and other isotonic liquids, including adjuvants, such as D-sorbitol, D-mannose, D-mannitol, and sodium chloride, can be used as aqueous solutions for injection. These can be used in conjunction with suitable solubilizers, such as alcohol, for example, ethanol; polyalcohols, such as propylene glycol and polyethylene glycol; and non-ionic surfactants, such as Polysorbate 80™ and HCO-50.

Sesame oil or soy-bean oil can be used as an oleaginous liquid, which may be used in conjunction with benzyl benzoate or benzyl alcohol as a solubilizer, and may be formulated with a buffer, such as phosphate buffer and sodium acetate buffer; a pain-killer, such as procaine hydrochloride; a stabilizer, such as benzyl alcohol and phenol; and/or an anti-oxidant. A prepared injection may be filled into a suitable ampoule.

Formulations for rectal administration include suppositories with standard carriers such as cocoa butter or polyethylene glycol. Formulations for topical administration in the mouth, for example, buccally or sublingually, include lozenges, which contain the active ingredient in a flavored base such as sucrose and acacia or tragacanth, and pastilles including the active ingredient in a base such as gelatin, glycerin, sucrose or acacia. For intra-nasal administration of an active ingredient, a liquid spray or dispersible powder or in the form of drops may be used. Drops may be formulated with an aqueous or non-aqueous base also including one or more dispersing agents, solubilizing agents or suspending agents.

For administration by inhalation the compositions are conveniently delivered from an insufflator, nebulizer, pressurized packs or other convenient means of delivering an aerosol spray. Pressurized packs may include a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the compositions may take the form of a dry powder composition, for example, a powder mix of an active ingredient and a suitable powder base such as lactose or starch. The powder composition may be presented in unit dosage form in, for example, capsules, cartridges, gelatin or blister packs from which the powder may be administered with the aid of an inhalator or insufflators.

Other formulations include implantable devices and adhesive patches; which release a therapeutic agent.

When desired, the above-described formulations, adapted to give sustained release of the active ingredient, may be employed. The pharmaceutical compositions may also contain other active ingredients such as antimicrobial agents, immunosuppressants or preservatives.

It should be understood that in addition to the ingredients particularly mentioned above, the formulations of this invention may include other agents conventional in the art having regard to the type of formulation in question, for example, those suitable for oral administration may include flavoring agents.

Preferred unit dosage formulations are those containing an effective dose, as recited below under the item of ‘Method for treating or preventing cancer’, of each of the active ingredients of the present invention or an appropriate fraction thereof.

### V-1. Pharmaceutical Compositions Including Screened Agents

The present invention provides compositions for treating or preventing cancers including any of the agents selected by the above-described screening methods of the present invention.

An agent identified by a method of the present invention can be directly administered or can be formulated into a dosage form according to any conventional pharmaceutical preparation method detailed above.

### V-2. Pharmaceutical Compositions Including Double-Stranded Molecule

The present invention provides compositions for treating or preventing cancers including any of the double-stranded molecules described above in item ‘I. Double-stranded molecule’ or selected by the above-described screening methods of the present invention.

A double-stranded molecule of the present invention can be adapted for use to prevent or treat cancers which overexpressing IMP-1 gene, such as lung cancers, e.g. NSCLC.

In one embodiment, a composition comprising one or more double-stranded molecules of the invention can be encapsulated in a delivery vehicle, e.g. liposomes, for administration to a subject, carriers and diluents and their salts, and/or can be present in pharmaceutically acceptable formulations. Methods for the delivery of nucleic acid molecules are described in Akhtar S & Juliano R L. Trends Cell Biol. 1992 May; 2(5):139-44.; Delivery Strategies for Antisense Oligonucleotide Therapeutics, ed. Akhtar, 1995; Maurer N, et al., Mol Membr Biol. 1999 January-March; 16(1):129-40.; Hofland & Huang. Handb Exp Pharmacol. 1999 137:165-192. It further describe the general methods for delivery of nucleic acid molecules (U.S. Pat. No. 6,395,713 and WO 199402595). These protocols can be utilized for the delivery of virtually any double-stranded molecule. Double-stranded molecules can be administered to cells by a variety of methods known to those of skill in the art, including but not restricted to, encapsulation in liposomes, by iontophoresis, or by incorporation into other vehicles, such as biodegradable polymers, hydrogels, cyclodextrins (see for example Gonzalez H, et al., Bioconjug Chem. 1999 November-December; 10(6):1068-74.; WO 03/47518 and WO 03/46185), poly (lactic-co-glycolic) acid (PLGA) and PLCA microspheres (see for example U.S. Pat. No. 6,447,796 and US 2002130430), biodegradable nanocapsules, and bioadhesive microspheres, or by proteinaceous vectors (WO 200053722). In another embodiment, the nucleic acid molecules of the invention can also be formulated or complexed with polyethyleneimine and derivatives thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives. In one embodiment, the nucleic acid molecules of the invention are formulated as described in US 20030077829 (i.e. lipid-based formulations), incorporated by reference herein in its entirety.

The double-stranded molecules of the present invention can also be administered to a subject in combination with other therapeutic compounds to increase the overall therapeutic effect. The use of multiple compounds to treat an indication can increase the beneficial effects while reducing the presence of side effects.

### V-3. Pharmaceutical Compositions Including Antisense Nucleic Acids

Antisense nucleic acids corresponding to the nucleotide sequence of the IMP-1 gene can be used to reduce the expression level of the gene, which is up-regulated in various cancerous cells, are useful for the treatment of cancer and thus are also encompassed by the present invention. An antisense nucleic acid acts by binding to the nucleotide sequence of the IMP-1 gene, or mRNAs corresponding thereto, thereby inhibiting the transcription or translation of the gene, promoting the degradation of the mRNAs, and/or inhibiting the expression of the protein encoded by the gene. Thus, as a result, an antisense nucleic acid inhibits the IMP-1 protein to function in the cancerous cell. Herein, the phrase “antisense nucleic acids” refers to nucleotides that specifically hybridize to a target sequence and includes not only nucleotides that are entirely complementary to the target sequence but also that includes mismatches of one or more nucleotides. For example, the antisense nucleic acids of the present invention include polynucleotides that have a homology of at least 70% or higher, preferably of at least 80% or higher, more preferably of at least 90% or higher, even more preferably of at least 95% or higher over a span of at least 15 continuous nucleotides of the IMP-1 gene or the complementary sequence thereof. Algorithms known in the art can be used to determine such homology.

Antisense nucleic acids of the present invention act on cells producing proteins encoded by the IMP-1 gene by binding to the DNA or mRNA of the gene, inhibiting their transcription or translation, promoting the degradation of the mRNA, and inhibiting the expression of the protein, finally inhibiting the protein to function.

Antisense nucleic acids of the present invention can be made into an external preparation, such as a liniment or a poultice, by admixing it with a suitable base material which is inactive against the nucleic acids.

Also, as needed, the antisense nucleic acids of the present invention can be formulated into tablets, powders, granules, capsules, liposome capsules, injections, solutions, nose-drops and freeze-drying agents by adding excipients, isotonic agents, solubilizers, stabilizers, preservatives, pain-killers, and such. An antisense-mounting medium can also be used to increase durability and membrane-permeability. Examples include, but are not limited to, liposomes, poly-L-lysine, lipids, cholesterol, lipofectin, or derivatives of these. These can be prepared by following known methods.

The antisense nucleic acids of the present invention inhibit the expression of the IMP-1 protein and are useful for suppressing the biological activity of the protein. In addition, expression-inhibitors, including antisense nucleic acids of the present invention, are useful in that they can inhibit the biological activity of the IMP-1 protein.

The antisense nucleic acids of present invention include modified oligonucleotides. For example, thioated oligonucleotides may be used to confer nuclease resistance to an oligonucleotide.

### V-4. Pharmaceutical Compositions Including Antibodies

The function of a gene product of the IMP-1 gene which is over-expressed in various cancers can be inhibited by administering a compound that binds to or otherwise inhibits the function of the gene products. An antibody against the IMP-1 polypeptide can be mentioned as such a compound and can be used as the active ingredient of a pharmaceutical composition for treating or preventing cancer.

The present invention relates to the use of antibodies against a protein encoded by the IMP-1 gene, or fragments of the antibodies. As used herein, the term “antibody” refers to item of II. Antibody.

An antibody may be modified by conjugation with a variety of molecules, such as polyethylene glycol (PEG). The present invention includes such modified antibodies. The modified antibody can be obtained by chemically modifying an antibody. Such modification methods are conventional in the field.

Alternatively, the antibody used for the present invention may be a chimeric antibody having a variable region derived from a non-human antibody against the IMP-1 polypeptide and a constant region derived from a human antibody, or a humanized antibody, composed of a complementarity determining region (CDR) derived from a non-human antibody, a frame work region (FR) and a constant region derived from a human antibody. Such antibodies can be prepared by using known technologies. Humanization can be performed by substituting rodent CDRs or CDR sequences for the corresponding sequences of a human antibody (see e.g., Verhoeyen et al., Science 1988, 239:1534-6). Accordingly, such humanized antibodies are chimeric antibodies, wherein substantially less than an intact human variable domain has been substituted by the corresponding sequence from a non-human species.

Complete human antibodies including human variable regions in addition to human framework and constant regions can also be used. Such antibodies can be produced using various techniques known in the art. For example in vitro methods involve use of recombinant libraries of human antibody fragments displayed on bacteriophage (e.g., Hoogenboom et al., J Mol Biol 1992, 227:381-8). Similarly, human antibodies can be made by introducing human immunoglobulin loci into transgenic animals, e.g., mice in which the endogenous immunoglobulin genes have been partially or completely inactivated. This approach is described, e.g., in U.S. Pat. Nos. 6,150,584; 5,545,807; 5,545,806; 5,569,825; 5,625,126; 5,633,425; and 5,661,016.

When the obtained antibody is to be administered to the human body (antibody treatment), a human antibody or a humanized antibody is preferable for reducing immunogenicity.

Antibodies obtained as above may be purified to homogeneity. For example, the separation and purification of the antibody can be performed according to separation and purification methods used for general proteins. For example, the antibody may be separated and isolated by the appropriately selected and combined use of column chromatographies, such as affinity chromatography, filter, ultrafiltration, salting-out, dialysis, SDS polyacrylamide gel electrophoresis, isoelectric focusing, and others (Antibodies: A Laboratory Manual. Ed Harlow and David Lane, Cold Spring Harbor Laboratory (1988)), but are not limited thereto. A protein A column and protein G column can be used as the affinity column. Exemplary protein A columns to be used include, for example, Hyper D, POROS, and Sepharose F.F. (Pharmacia).

Exemplary chromatography, with the exception of affinity includes, for example, ion-exchange chromatography, hydrophobic chromatography, gel filtration, reverse-phase chromatography, adsorption chromatography, and the like (Strategies for Protein Purification and Characterization: A Laboratory Course Manual. Ed Daniel R. Marshak et al., Cold Spring Harbor Laboratory Press (1996)). The chromatographic procedures can be carried out by liquid-phase chromatography, such as HPLC and FPLC.

### VI. Methods for Treating or Preventing Cancer:

Cancer therapies directed at specific molecular alterations that occur in cancer cells have been validated through clinical development and regulatory approval of anti-tumor pharmaceuticals such as trastuzumab (Herceptin) for the treatment of advanced cancers, imatinib mesylate (Gleevec) for chronic myeloid leukemia, gefitinib (Iressa) for non-small cell lung cancer (NSCLC), and rituximab (anti-CD20 mAb) for B-cell lymphoma and mantle cell lymphoma (Ciardiello F et al., Clin Cancer Res 2001, 7:2958-70, Review; Slamon D J et al., N Engl J Med 2001, 344:783-92; Rehwald U et al., Blood 2003, 101:420-4; Fang Get al., Blood 2000, 96:2246-53). These drugs are clinically effective and better tolerated than traditional anti-tumor agents because they target only transformed cells. Hence, such drugs not only improve survival and quality of life for cancer patients, but also validate the concept of molecularly targeted cancer therapy. Furthermore, targeted drugs can enhance the efficacy of standard chemotherapy when used in combination with it (Gianni L, Oncology 2002, 63 Suppl 1:47-56; Klejman A et al., Oncogene 2002, 21:5868-76). Therefore, future cancer treatments will probably involve combining conventional drugs with target-specific agents aimed at different characteristics of tumor cells such as angiogenesis and invasiveness.

These modulatory methods can be performed ex vivo or in vitro (e.g., by culturing the cell with the agent) or, alternatively, in vivo (e.g., by administering the agent to a subject). The methods involve administering a protein, or combination of proteins, or a nucleic acid molecule, or combination of nucleic acid molecules, as therapy to counteract aberrant expression of the differentially expressed genes or aberrant activity of their gene products.

Diseases and disorders that are characterized by increased (relative to a subject not suffering from the disease or disorder) expression levels or biological activities of genes and gene products, respectively, may be treated with therapeutics that antagonize (i.e., reduce or inhibit) activity of the over-expressed gene. Therapeutics that antagonize activity can be administered therapeutically or prophylactically.

Accordingly, therapeutics that may be utilized in the context of the present invention include, e.g., (i) a polypeptide of the over-expressed IMP-1 gene or analogs, derivatives, fragments or homologs thereof; (ii) antibodies to the over-expressed gene or gene products; (iii) nucleic acids encoding the over-expressed gene; (iv) antisense nucleic acids or nucleic acids that are “dysfunctional” (i.e., due to a heterologous insertion within the nucleic acids of over-expressed gene); (v) small interfering RNA (siRNA); or (vi) modulators (i.e., inhibitors, antagonists that alter the interaction between an over-expressed polypeptide and its binding partner). The dysfunctional antisense molecules are utilized to “knockout” endogenous function of a polypeptide by homologous recombination (see, e.g., Capecchi, Science 1989, 244: 1288-92).

Increased levels can be readily detected by quantifying peptide and/or RNA, by obtaining a patient tissue sample (e.g., from biopsy. tissue) and assaying it in vitro for RNA or peptide levels, structure and/or activity of the expressed peptides (or mRNAs of a gene whose expression is altered). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

Prophylactic administration occurs prior to the manifestation of overt clinical symptoms of disease, such that a disease or disorder is prevented or, alternatively, delayed in its progression. In the context of the present invention, prevention is any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications. Accordingly, the present invention encompasses a wide range of prophylactic therapies aimed at alleviating the severity of cancer, particularly lung cancer.

Therapeutic methods of the present invention may include the step of contacting a cell with an agent that modulates one or more of the activities of the IMP-1 gene products. Examples of agent that modulates protein activity include, but are not limited to, nucleic acids, proteins, naturally occurring cognate ligands of such proteins, peptides, peptidomimetics, and other small molecule.

Thus, the present invention provides methods for treating or alleviating a symptom of cancer, or preventing cancer in a subject by decreasing the expression of the IMP-1 gene or the activity of the gene product. The present method is particularly suited for treating or preventing NSCLCs.

Suitable therapeutics can be administered prophylactically or therapeutically to a subject suffering from or at risk of (or susceptible to) developing cancers. Such subjects can be identified by using standard clinical methods or by detecting an aberrant expression level (“up-regulation” or “over-expression”) of the IMP-1 gene or aberrant activity of the gene product.

According to an aspect of the present invention, an agent screened through the present method may be used for treating or preventing cancer. Methods well known to those skilled in the art may be used to administer the agents to patients, for example, as an intra-arterial, intravenous, or percutaneous injection or as an intranasal, transbronchial, intramuscular, or oral administration. If said agent is encodable by a DNA, the DNA can be inserted into a vector for gene therapy and the vector administered to a patient to perform the therapy.

The dosage and methods for administration vary according to the body-weight, age, sex, symptom, condition of the patient to be treated and the administration method; however, one skilled in the art can routinely select suitable dosage and administration method.

For example, although the dose of an agent that binds to the IMP-1 polypeptide and regulates the activity of the polypeptide depends on the aforementioned various factors, the dose is generally about 0.1 mg to about 100 mg per day, preferably about 1.0 mg to about 50 mg per day and more preferably about 1.0 mg to about 20 mg per day, when administered orally to a normal adult human (60 kg weight).

When administering the agent parenterally, in the form of an injection to a normal adult human (60 kg weight), although there are some differences according to the patient, to target organ, symptoms and methods for administration, it is convenient to intravenously inject a dose of about 0.01 mg to about 30 mg per day, preferably about 0.1 to about 20 mg per day and more preferably about 0.1 to about 10 mg per day. In the case of other animals, the appropriate dosage amount may be routinely calculated by converting to 60 kg of body-weight.

Similarly, a pharmaceutical composition of the present invention may be used for treating or preventing cancer. Methods well known to those skilled in the art may be used to administer the compositions to patients, for example, as an intra-arterial, intravenous, or percutaneous injection or as an intranasal, transbronchial, intramuscular, or oral administration.

For each of the aforementioned conditions, the compositions, e.g., polypeptides and organic compounds, can be administered orally or via injection at a dose ranging from about 0.1 to about 250 mg/kg per day. The dose range for adult humans is generally from about 5 mg to about 17.5 g/day, preferably about 5 mg to about 10 g/day, and most preferably about 100 mg to about 3 g/day. Tablets or other unit dosage forms of presentation provided in discrete units may conveniently contain an amount which is effective at such dosage or as a multiple of the same, for instance, units containing about 5 mg to about 500 mg, usually from about 100 mg to about 500 mg.

The dose employed will depend upon a number of factors, including the age, body weight and sex of the subject, the precise disorder being treated, and its severity. Also the route of administration may vary depending upon the condition and its severity. In any event, appropriate and optimum dosages may be routinely calculated by those skilled in the art, taking into consideration the above-mentioned factors.

In particular, an antisense nucleic acids against the IMP-1 gene can be given to the patient by direct application onto the ailing site or by injection into a blood vessel so that it will reach the site of ailment.

The dosage of the antisense nucleic acid derivatives of the present invention can be adjusted suitably according to the patient's condition and used in desired amounts. For example, a dose range of 0.1 to 100 mg/kg, preferably 0.1 to 50 mg/kg can be administered.

Hereinafter, the present invention is described in more detail with reference to the Examples. However, the following materials, methods and examples only illustrate aspects of the invention and in no way are intended to limit the scope of the present invention. As such, methods and materials similar or equivalent to those described herein can be used in the practice or testing of the present invention.

### VII. Method for Assessing the Prognosis of Lung Cancer

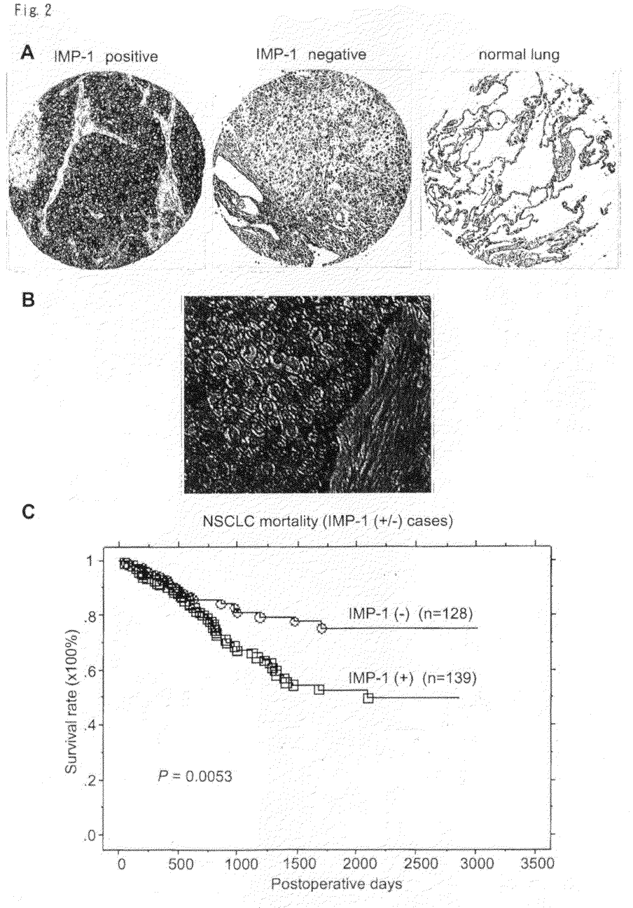

According to the present invention, it was newly discovered that IMP-1 expression is significantly associated with poorer prognosis of NSCLC patients (see FIG. 2C). Thus, the present invention provides a method for assessing or determining the prognosis of a patient with lung cancer, in particular, NSCLC, by detecting the expression level of the IMP-1 gene in a biological sample of the patient; comparing the detected expression level to a control level; and determining a increased expression level to the control level as indicative of poor prognosis (poor survival).

Herein, the term “prognosis” refers to a forecast as to the probable outcome of the disease as well as the prospect of recovery from the disease as indicated by the nature and symptoms of the case. Accordingly, a less favorable, negative, poor prognosis is defined by a lower post-treatment survival term or survival rate. Conversely, a positive, favorable, or good prognosis is defined by an elevated post-treatment survival term or survival rate.

In the context of the present invention, the phrase “assessing (or determining) the prognosis” is intended to encompass predictions and likelihood analysis of lung cancer, progression, particularly NSCLC recurrence, metastatic spread and disease relapse. The present method for assessing or determining prognosis is intended to be used clinically in making decisions concerning treatment modalities, including therapeutic intervention, diagnostic criteria such as disease staging, and disease monitoring and surveillance for metastasis or recurrence of neoplastic disease.

The patient-derived biological sample used for the method may be any sample derived from the subject to be assessed so long as the IMP-1 gene can be detected in the sample. Preferably, the biological sample is a lung cell (a cell obtained from the lung). Other suitable biological samples include, but are not limited to, bodily fluids such as sputum, blood, serum, or plasma. Alternatively, the sample may be cells purified from a tissue. The biological samples may be obtained from a patient at various time points, including before, during, and/or after a treatment.

According to the present invention, it was shown that the higher the expression level of the IMP-1 gene measured in the patient-derived biological sample, the poorer the prognosis for post-treatment remission, recovery, and/or survival and the higher the likelihood of poor clinical outcome. Thus, according to the present method, the “control level” used for comparison may be, for example, the expression level of the IMP-1 gene detected before any kind of treatment in an individual or a population of individuals who showed good or positive prognosis of NSCLC after the treatment, which herein will be referred to as “good prognosis control level”. Alternatively, the “control level” may be, for example, the expression level of the IMP-1 gene detected before any kind of treatment in an individual or a population of individuals who showed poor or negative prognosis of NSCLC after the treatment, which herein will be referred to as “poor prognosis control level”. The “control level” is a single expression pattern derived from a single reference population or from a plurality reference population. Thus, the control level may be determined based on the expression level of the IMP-1 gene detected before any kind of treatment in a patient of NSCLC, or a population of the patients whose disease state (good or poor prognosis) is known. It is preferred, to use the standard value of the expression levels of the IMP-1 gene in a patient group with a known disease state. The standard value may be obtained by any method known in the art. For example, a range of mean±2 S.D. or mean±3 S.D. may be used as standard value.

The control level may be determined at the same time with the test biological sample by using a sample(s) previously collected and stored before any kind of treatment from lung cancer patient(s) (control or control group) whose disease state (good prognosis or poor prognosis) are known.

Alternatively, the control level may be determined by a statistical method based on the results obtained by analyzing the expression level of the IMP-1 gene in samples previously collected and stored from a control group. Furthermore, the control level can be a database of expression patterns from previously tested cells. Moreover, according to an aspect of the present invention, the expression level of the IMP-1 gene in a biological sample may be compared to multiple control levels, which control levels are determined from multiple reference samples. It is preferred to use a control level determined from a reference sample derived from a tissue type similar to that of the patient-derived biological sample.

According to the present invention, a similarity in the expression level of the IMP-1 gene to the good prognosis control level indicates a more favorable prognosis of the patient and an increase in the expression level to the good prognosis control level indicates less favorable, poorer prognosis for post-treatment remission, recovery, survival, and/or clinical outcome. On the other hand, a decrease in the expression level of the IMP-1 gene to the poor prognosis control level indicates a more favorable prognosis of the patient and a similarity in the expression level to the poor prognosis control level indicates less favorable, poorer prognosis for post-treatment remission, recovery, survival, and/or clinical outcome.

An expression level of the IMP-1 gene in a biological sample can be considered altered when the expression level differs from the control level by more than 1.0, 1.5, 2.0, 5.0, 10.0, or more fold. Alternatively, an expression level of the IMP-1 gene in a biological sample can be considered altered, when the expression level is increased or decreased to the control level at least 10%, 20%, 30%, 40%, 50%, 60%, 80%, 90%, or more.

The difference in the expression level between the test biological sample and the control level can be normalized to a control, e.g., housekeeping gene. For example, polynucleotides whose expression levels are known not to differ between the cancerous and non-cancerous cells, including those coding for β-actin, glyceraldehyde 3-phosphate dehydrogenase, and ribosomal protein P1, may be used to normalize the expression levels of the IMP-1 gene.

The expression level may be determined by detecting the gene transcript in the patient-derived biological sample using techniques well known in the art. The gene transcripts detected by the present method include both the transcription and translation products, such as mRNA and protein.

For instance, the transcription product of the IMP-1 gene can be detected by hybridization, e.g., Northern blot hybridization analyses, that use an IMP-1 gene probe to the gene transcript. The detection may be carried out on a chip or an array. The use of an array is preferable for detecting the expression level of a plurality of genes including the IMP-1 gene. As another example, amplification-based detection methods, such as reverse-transcription based polymerase chain reaction (RT-PCR) which use primers specific to the IMP-1 gene may be employed for the detection (see Example). The IMP-1 gene-specific probe or primers may be designed and prepared using conventional techniques by referring to the whole sequence of the IMP-1 gene (SEQ ID NO: 11). For example, the primers (SEQ ID NOs: 1, 2, 3 and 4) used in the Example may be employed for the detection by RT-PCR, but the present invention is not restricted thereto.

Specifically, a probe or primer used for the present method hybridizes under stringent, moderately stringent, or low stringent conditions to the mRNA of the IMP-1 gene. As used herein, the phrase “stringent (hybridization) conditions” refers to conditions under which a probe or primer will hybridize to its target sequence, but to no other sequences. Stringent conditions are sequence-dependent and will be different under different circumstances. Specific hybridization of longer sequences is observed at higher temperatures than shorter sequences. Generally, the temperature of a stringent condition is selected to be about 5° C. lower than the thermal melting point (Tₘ) for a specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes or primers (e.g., 10 to 50 nucleotides) and at least about 60° C. for longer probes or primers. Stringent conditions may also be achieved with the addition of destabilizing agents, such as formamide.

Alternatively, the translation product may be detected for the assessment of the present invention. For example, the quantity of the IMP-1 protein may be determined. A method for determining the quantity of the protein as the translation product includes immunoassay methods that use an antibody specifically recognizing the IMP-1 protein. The antibody may be monoclonal or polyclonal. Furthermore, any fragment or modification (e.g., chimeric antibody, scFv, Fab, F(ab′)₂, Fv, etc.) of the antibody may be used for the detection, so long as the fragment retains the binding ability to the IMP-1 protein. Methods to prepare these kinds of antibodies for the detection of proteins are well known in the art, and any method may be employed in the present invention to prepare such antibodies and equivalents thereof.

Alternatively, the expression level of the IMP-1 gene may be determined from the intensity of staining observed via immunohistochemical analysis using an antibody against IMP-1 protein. Namely, the observation of strong staining indicates increased presence of the IMP-1 protein and at the same time high expression level of the IMP-1 gene. NSCLC tissue can be preferably used as a test material for immunohistochemical analysis.

Moreover, in addition to the expression level of the IMP-1 gene, the expression level of other lung cell-associated genes, for example, genes known to be differentially expressed in NSCLC, may also be determined to improve the accuracy of the assessment. Such other lung cell-associated genes include those described in WO 2004/031413 and WO 2005/090603.

The patient to be assessed for the prognosis of NSCLC according to the method is preferably a mammal and includes human, non-human primate, mouse, rat, dog, cat, horse, and cow.

Unless otherwise defined, all technical and scientific terms used herein have the same meaning as commonly understood by one of ordinary skill in the art to which this invention belongs. In case of conflict, the present specification, including definitions, will control.

Hereinafter, the present invention is described in more detail with reference to the Examples. However, the following materials, methods and examples only illustrate aspects of the invention and in no way are intended to limit the scope of the present invention. As such, methods and materials similar or equivalent to those described herein can be used in the practice or testing of the present invention.

### Examples

### I. Materials and Methods

A. Lung-Cancer Cell Lines and Clinical Samples

The human lung-cancer used herein were as follows: adenocarcinomas (ADC), A427, A549, LC319, PC3, PC9, PC14, and NCI-H1373; bronchioloalveolar-cell carcinomas (BAC), NCI-H1666 and NCI-H1781; adenosquamous carcinomas (ASC), NCI-H226 and NCI-H647; lung squamous-cell carcinomas (SCC), RERF-LC-AI, SK-MES-1, EBC-1, LU61, NCI-H520, NCI-H1703, and NCI-H2170; a lung large-cell carcinoma (LCC) LX1; small-cell lung cancers (SCLC), DMS114, DMS273, SBC-3, and SBC-5. All cells were grown in monolayer in appropriate medium supplemented with 10% fetal calf serum (FCS) and were maintained at 37° C. in atmospheres of humidified air with 5% CO₂. Human small airway epithelial cells (SAEC) were grown in optimized medium (SAGM) purchased from Cambrex Bio Science Inc. (Walkersville, Md.). 14 primary NSCLCs (seven ADCs and seven SCCs) were obtained along with adjacent normal lung-tissue samples.

A total of 267 formalin-fixed primary NSCLCs (stage I-IIIA) and adjacent normal lung tissue samples used for immunostaining on tissue microarrays had been obtained with informed consent from patients undergoing curative surgical operation. Histological classification of tumors was done according to the World Health Organization criteria (International Histological Classification of Tumours, 3rd edition. Genova: World Health Organization, 1999.). All tumors were staged on the basis of the pTNM pathological classification of the UICC (International Union Against Cancer) (Sobin, L., et al. New York: Wiley-Liss, Inc., 2002.). Postoperative staging evaluation demonstrated that 101 patients were at stage IA, 88 at stage IB, 8 at stage IIA, 27 at stage IIB, and 43 at stage Histopathological examination of resected tumors revealed that 157 cases were diagnosed as ADC, 93 cases as SCCs, 13 as LCCs, and 4 as ASCs (Table 1). This study as well as the use of all clinical materials described above were approved by individual institutional Ethical Committees.

TABLE 1
Associations Between IMP-1 Expressions and
Clinicopathological Features in Patients with Lung Cancer

| | | IMP-1 EXPRESSION | | |
|---|---|---|---|---|
| | | positive | negative | |
| variables | No. of cases | (n = 139) | (n = 128) | P-value |
| Age (year) | | | | |
| <60 | 84 | 38 | 46 | ¹P = 0.1306 |
| ≧60 | 183 | 101 | 82 | |
| Gender | | | | |
| Male | 177 | 107 | 70 | ¹P = 0.0001* |
| Female | 90 | 32 | 58 | |
| pT | | | | |
| pT1 | 117 | 43 | 74 | ²P = 0.0003* |
| pT2 | 123 | 80 | 43 | |
| pT3 | 27 | 16 | 11 | |
| pN | | | | |
| pN0 | 205 | 100 | 105 | ²P = 0.1639 |
| pN1 | 26 | 17 | 9 | |
| pN2 | 36 | 22 | 14 | |
| Histology | | | | |
| ^{a}ADC | 157 | 57 | 100 | ¹P < 0.0001* |
| (including ^{b}BAC) | | | | |
| ^{c}SCC | 93 | 70 | 23 | |
| ^{d}LCC | 13 | 10 | 3 | |
| ^{e}ASC | 4 | 2 | 2 | |
| Tumor grade | | | | |
| G1 | 79 | 27 | 52 | ¹P = 0.0001* |
| Other | 188 | 112 | 76 | |

B. Semiquantitative RT-PCR

Total RNA was extracted from cultured cells and clinical tissues using Trizol reagent (Life Technologies, Inc., Gaithersburg, Md.) according to the manufacturer's protocol. Extracted RNAs and normal human tissue poly(A) RNAs were treated with DNase I (Nippon Gene, Tokyo, Japan) and reversely-transcribed using oligo (dT) primer and SuperScript H reverse transcriptase (Invitrogen, Carlsbad, Calif.). Semiquantitative RT-PCR experiments were carried out with the following synthesized IMP-1-specific primers or with β-actin (ACTB)-specific primers as an internal control:

| | |
|---|---|
| IMP-1 | |
| 5′-CAGAAGGGACAGAGTAACCAG-3′ | (SEQ ID NO: 1) |
| and | |
| 5′-GAGATCAGGGTTCCTCACTG-3′; | (SEQ ID NO: 2) |
| ACTB, | |
| 5′-GAGGTGATAGCATTGCTTTCG-3′ | (SEQ ID NO: 3) |
| 5′-CAAGTCAGTGTACAGGTAAGC-3′. | (SEQ ID NO: 4) |

PCR reactions were optimized for the number of cycles to ensure product intensity within the logarithmic phase of amplification.

C. Northern-Blot Analysis

Human multiple-tissue blots (BD Biosciences Clontech, Palo Alto, Calif.) were hybridized with a ³²P-labeled PCR product of IMP-1. The cDNA probes of IMP-1 were prepared by RT-PCR using the primers described above. Pre-hybridization, hybridization, and washing were performed according to the supplier's recommendations. The blots were autoradiographed at room temperature for 30 hours with intensifying BAS screens (BIO-RAD, Hercules, Calif.).

D. Preparation of Anti-IMP-1 Polyclonal Antibody

Rabbit antibodies specific for IMP-1 were raised by immunizing rabbits with IMP-1 peptides (IEHSVPKKQRSRKIC (SEQ ID NO: 5) and CVKQQHQKGQSNQAQARRK (SEQ ID NO: 6)), and purified using standard protocols. The antibody was confirmed by western blot to be specific to IMP-1, and to not cross-react with other homologous proteins, IMP-2 and IMP-3 using lysates from NSCLC cell lines transfected with IMP-1, -2, and -3 expressing vector and those from endogenous IMP-1 expressing/non-expressing NSCLC cells.

E. Western-Blot Analysis

Cells were lysed in lysis buffer; 50 mM Tris-HCl (pH 8.0), 150 mM NaCl, 0.5% NP-40, 0.5% deoxycholate-Na, 0.1% SDS, plus protease inhibitor (Protease Inhibitor Cocktail Set III; Calbiochem Darmstadt, Germany). An ECL western-blotting analysis system (GE Healthcare Bio-sciences, Piscataway, N.J.), as previously described (Kato, T., et al. Cancer Res, 65: 5638-5646, 2005.; Furukawa, C., et al. Cancer Res, 65: 7102-7110, 2005.; Suzuki, C., et al. Cancer Res, 65: 11314-11325, 2005.) was used. SDS-PAGE was performed in 7.5% polyacrylamide gels. PAGE-separated proteins were electroblotted onto nitrocellulose membranes (GE Healthcare Bio-sciences) and incubated with a rabbit polyclonal anti-human IMP-1 antibody. A goat anti-rabbit IgG-HRP antibody (GE Healthcare Bio-sciences) was served as the secondary antibodies for these experiments.

F. Tissue Microarray Construction and Immunohistochemistry

Lung cancer tissue microarrays were constructed as published elsewhere, using formalin-fixed NSCLCs (Ishikawa, N., et al. Clin Cancer Res, 10: 8363-8370, 2004.; Kato, T., et al. Cancer Res, 65: 5638-5646, 2005.; Furukawa, C., et al. Cancer Res, 65: 7102-7110, 2005.; Suzuki, C., et al. Cancer Res, 65: 11314-11325, 2005.). Tissue areas for sampling were selected based on visual alignment with the corresponding HE-stained sections on slides. Three, four, or five tissue cores (diameter 0.6 mm; height 3-4 mm) taken from donor-tumor blocks were placed into recipient paraffin blocks using a tissue microarrayer (Beecher Instruments, Sun Prairie, Wis.). A core of normal tissue area was punched from each case. Five-μm sections of the resulting microarray block were used for immunohistochemical analysis.

To investigate the IMP-1 protein level in tissue microarrays of clinical samples, sections were stained using ENVISION+ Kit/HRP (DakoCytomation, Glostrup, Denmark). A rabbit polyclonal anti-IMP-1 antibody was added after blocking endogenous peroxidase and proteins, and the sections were incubated with HRP-labeled anti-rabbit IgG as the secondary antibody. Substrate-chromogen was added and the specimens were counterstained with hematoxylin. Positivity for IMP-1 was assessed semiquantitatively by three independent investigators without prior knowledge of the clinical follow-up data, each of whom recorded staining intensity as negative (scored as 0), or positive (1+). Cases were accepted as positive only if reviewers independently defined them as such.

G. Statistical Analysis

All analyses were performed using statistical analysis software (StatView, version 5.0; SAS Institute, Inc. Cary, N.C., USA). Clinicopathological variables, such as age, gender, pathological TNM stage, histological type, and histopathological grading, were correlated with the expression levels of IMP-1 protein determined by tissue-microarray analysis. IMP-1 immunoreactivity was assessed for association with clinicopathologic variables using the following statistical tests, such as the Mann-Whitney U-test or chi-square test. Tumor-specific survival curves were calculated from the date of surgery to the time of death related to NSCLC, or to the last follow-up observation. The Kaplan-Meier method was used to generate survival curves, and survival differences were analyzed with the log-rank test, based on the status of IMP-1 expression. Univariate analysis was performed using Cox's proportional hazard regression model.

H. RNA Interference Assay

An vector-based RNA interference (RNAi) system, psiH1BX3.0, that was designed to synthesize siRNAs in mammalian cells was previously established (Kato, T., et al. Cancer Res, 65: 5638-5646, 2005.; Furukawa, C., et al. Cancer Res, 65: 7102-7110, 2005.; Suzuki, C., et al. Cancer Res, 65: 11314-11325, 2005.; Suzuki, C., et al. Cancer Res, 63: 7038-7041, 2003.). 10 μg of siRNA-expression vector was transfected using 30 μl of Lipofectamine 2000 (Invitrogen) into NSCLC cell lines, A549 and LC319. The transfected cells were cultured for seven days in the presence of appropriate concentrations of geneticin (G418), and the number of colonies was counted by colony-formation assay, and viability of the cells was evaluated by MTT assay 7 days after the treatment. In MTT assay, Cell-counting kit-8 solution (DOJINDO, Kumamoto, Japan) was added to each dish at a concentration of 1/10 volume, and the plates were incubated at 37° C. for additional 4 hours. Absorbance was then measured at 490 nm, and at 630 nm as a reference, with a Microplate Reader 550 (BIO-RAD). The target sequences of the synthetic oligonucleotides for RNAi were as follows: control 1 (EGFP: enhanced green fluorescent protein gene, a mutant of Aequorea victoria GFP), 5′-GAAGCAGCACGACTTCTTC-3′ (SEQ ID NO: 7); control 2 (Scramble: chloroplast Euglena gracilis gene coding for 5S and 16S rRNAs), 5′-GCGCGCTTTGTAGGATTCG-3′(SEQ ID NO:8); siRNA-IMP-1-#2,5′-GGAGGAGAACTTCTTTGGT-3′ (SEQ ID NO: 9); siRNA-IMP-1-#3,5′-GAATCTATGGCAAACTCAA-3′ (SEQ ID NO: 10). To validate the RNAi system, individual control siRNAs were tested by semiquantitative RT-PCR to confirm the decrease in expression of the corresponding target genes that had been transiently transfected to COS-7 cells. Down-regulation of IMP-1 expression by functional siRNA, but not by controls, was also confirmed in the cell lines used for this assay.

I. RNA-Immunoprecipitation and cDNA Microarray Screening for Identification of IMP-1-Associated mRNAs

The RNA immunoprecipitation protocol by Niranjanakumari et al. was utilized herein to analyze RNA(s)-protein interactions involving IMP-1 in vivo (Niranjanakumari, S., et al. Methods, 26: 182-190, 2002.). To determine the IMP-1 associated mRNA(s), IMP-1 constructs were transfected with NH2 (N)-terminal FLAG- or COOH (C)-terminal HA-tagged sequences (pCAGGS-n3FH-IMP-1 vector) into A549 cells. Using these cell lysates transfected with IMP-1 construct, immunoprecipitation experiments were further performed twice, first with monoclonal anti-FLAG M2 and then with monoclonal anti-HA antibody. A 2.5-μg aliquot of T7-based amplified mRNA (aRNAs) from each immunoprecipiated RNA (IP-RNA) and from the total RNA were reversely transcribed in the presence of Cy5-dCTP and Cy3-dCTP respectively as described previously (Kikuchi, T., et al. Oncogene, 22: 2192-2205, 2003.; Kakiuchi S, et al. Hum Mol Genet, 13: 3029-3043, 2004; Taniwaki M, et al. Int J Oncol, 2006 in press.), for hybridization to a cDNA microarray representing 27,648 genes (IP-microarray analysis).

II. Results

A. Expression of IMP-1 Transcripts in Lung Tumors and Normal Tissues

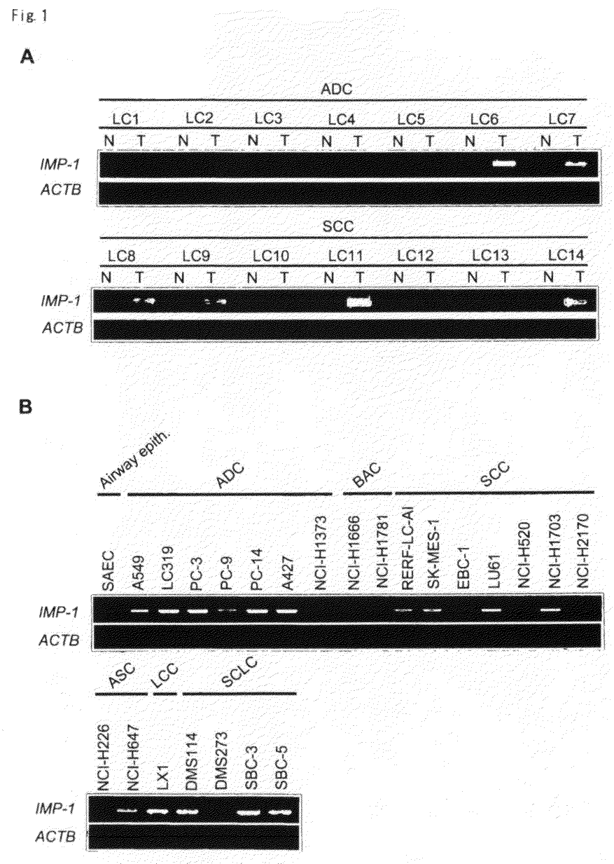

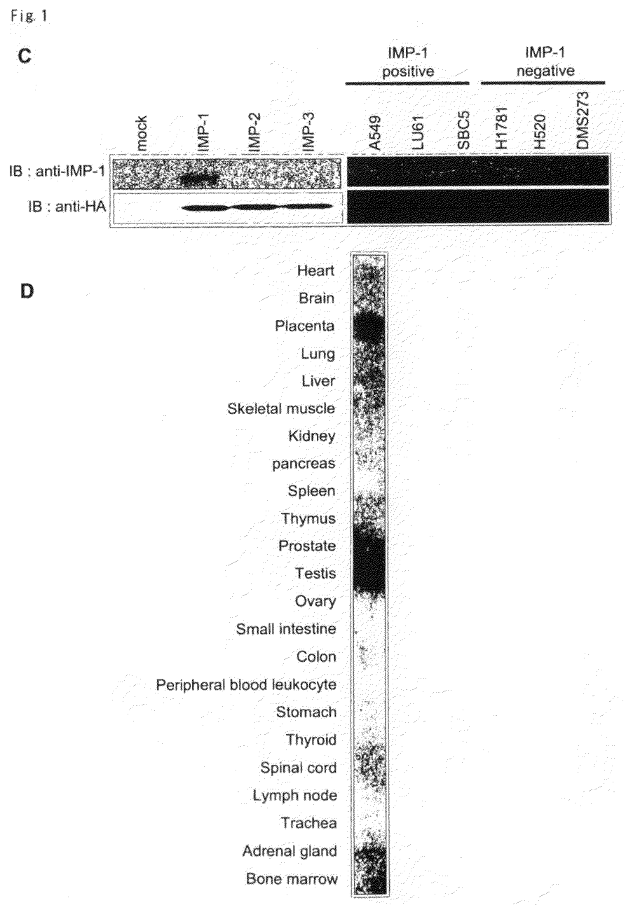

To identify target molecules for development of novel therapeutic agents and/or biomarkers for lung cancer, a cDNA microarray was first screened for genes that showed 5-fold or higher expression in more than 50% of NSCLCs analyzed (Kikuchi, T., et al. Oncogene, 22: 2192-2205, 2003.; Kakiuchi S, et al. Hum Mol Genet, 13: 3029-3043, 2004; Taniwaki M, et al. Int J Oncol, 2006 in press.). Among 27,648 genes screened, the IMP-1 transcript was identified to be over-expressed in the majority of NSCLCs; its over-expression was confirmed by semiquantitative RT-PCR experiments in 6 of 14 additional NSCLC cases (2 of 7 adenocarcinomas (ADCs) and 4 of 7 squamous-cell carcinomas (SCCs)) (FIG. 1A) as well as in 16 of 23 NSCLC and small-cell lung cancer (SCLC) cell lines. However, its expression was hardly detectable in SAEC cells derived from normal bronchial epithelium (FIG. 1B).

Rabbit polyclonal antibody against human IMP-1 was subsequently generated and its specificity to IMP-1 was confirmed by western-blot analysis that showed no cross-reactivity to other homologous proteins, IMP-2 and IMP-3, using lysate from NCI-H520 cells; the present inventors transfected HA-tagged IMP-1, -2, and -3 expression vector into the NCI-H520 cells that expressed neither of endogenous IMP-1, -2, and -3 (FIG. 1C_left). Using this antibody, expression of endogenous IMP-1 protein was confirmed in six lung-cancer cell lines by western blot analysis (three IMP-1-positive and three IMP-1-negative cell lines) (FIG. 1C_right). Northern-blot analysis using IMP-1 cDNA as a probe identified strong signals corresponding to 4.5-kb transcript that expressed abundantly specifically in placenta and testis (FIG. 1D).

B. Association of IMP-1 Expression with Poor Prognosis of NSCLC Patients

To verify the clinicopathological significance of IMP-1, the expression of IMP-1 protein was additionally examined by means of tissue microarrays containing lung-cancer tissues from 267 patients. Positive tumor cells generally showed a cytoplasmic staining pattern in NSCLC and no staining was observed in any of their adjacent normal lung tissues (FIG. 2A, B). Patterns of IMP-1 expression were classified as negative (scored as 0) or positive (scored as 1+) (FIG. 2A, B). Positive staining was found in 139 (52.1%) of 267 NSCLC cases; 57 of 157 ADC tumors (36.3%), 70 of 93 SCC tumors (75.3%), 10 of 13 LCC tumors (76.9%), and 2 of 4 ASC tumors (50.0%) were judged to be positive (Table 1). A correlation of IMP-1 expression was then examined with various clinicopathological parameters. A significant correlation with gender (higher in male; P=0.0001), pT classification (higher in larger tumor; P=0.0003), histopathological type (higher in non-ADC, P<0.0001), and histopathologic grade (higher in poorly differentiated tumor; P=0.0001) was found. No significant association was noted between IMP-1 expression and other clinicopathologic variables (Table 1).

The Kaplan-Meier analysis indicated a significant association between IMP-1-positivity in NSCLCs and tumor-specific 5-year survival (P=0.0053 by the Log-rank test) (FIG. 2C). By univariate analysis using the Cox proportional-hazard model, gender (male vs female), pT (T2-4 vs T1), pN (N1-2 vs NO), histopathological type (non-ADC vs ADC), and IMP-1 expression (positive vs negative) were all significantly related to poor tumor-specific survival among NSCLC patients (P=0.0286, <0.0001, <0.0001, 0.0003, and 0.0064, respectively; Table 2).

TABLE 2
Prognostic Factors in Cox's Proportional Hazards Model

| | | Univariate | |
|---|---|---|---|
| Variables | Risk ratio | 95% CI | P value |
| Age (year) | | | |
| ≧60/<60 | 1.686 | 0.963-2.954 | 0.0677 |
| Sex | | | |
| Male/Female | 1.848 | 1.066-3.205 | 0.0286* |
| pT | | | |
| pT2-4/pT1 | 3.145 | 1.789-5.525 | <0.0001* |
| pN | | | |
| pN1-2/pN0 | 4.310 | 2.660-6.993 | <0.0001* |
| Histological type | | | |
| non-ADC/ADC | 2.463 | 1.508-4.016 | 0.0003* |
| Histopathologic | | | |
| grade | | | |
| Other/G1 | 1.072 | 0.645-1.783 | 0.7879 |
| IMP-1 expression | | | |
| positive/negative | 2.045 | 1.224-3.425 | 0.0064* |

C. Growth Inhibition of NSCLC Cells by Specific siRNA Against IMP-1

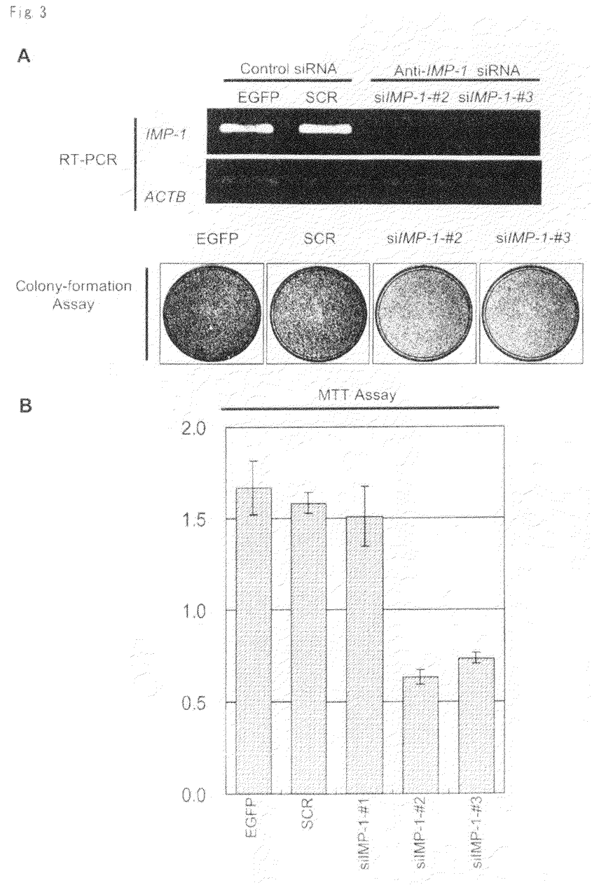

To assess whether IMP-1 is essential for growth or survival of lung-cancer cells, plasmids were constructed to express siRNAs against IMP-1 (si-IMP-#2, and -#3) as well as control plasmids (siRNAs for EGFP and Scramble) and transfected them into lung-cancer cell lines, A549 and LC319. The mRNA levels in cells transfected with si-IMP-1-#2 or -#3 were significantly decreased in comparison with cells transfected with either control siRNAs. Significant decreases were observed in the number of colonies and in the numbers of viable cells measured by MTT assay, suggesting that up-regulation of IMP-1 is related to growth or survival of cancer cells (representative data of A549 was shown in FIG. 3A, B).

D. Verification of the Clinicopathological Significance of IMP-1

IMP-1 protein is known to exhibit attachments to at least four RNAs (Ioannidis, P., et al. J Biol Chem, 280: 20086-93, 2005.). IMP-1 binds specifically to (1) one of the two cis-acting, c-myc mRNA instability elements (Bernstein, P. L., et al. Genes Dev, 6: 642-654, 1992.), (2) the 5′-untranslated region of the leader-3 IGF-II mRNA, which represents the major embryonic form of this message (Nielsen, J., et al. Mol Cell Biol, 19: 1262-70, 1999.), (3) the H19 RNA, a gene product exhibiting an oncofetal pattern of expression (Runge, S., et al. J Biol Chem, 275: 29562-9, 2000.), and (4) the neuron specific tau mRNA that encodes a microtubule-associated protein localized primarily in the cell body and axon of developing neurons (Atlas, R., et al. J Neurochem, 89: 613-26, 2004.). However, expression pattern of these mRNAs in lung cancers examined herein were not necessarily concordant with that of IMP-1 (data not shown). Therefore, to elucidate the function of IMP-1 in pulmonary carcinogenesis, other candidate mRNA(s) that would interact with IMP-1 and might thereby play important roles in growth and/or progression of lung cancer using RNA-immunoprecipitation and cDNA microarray (IP-microarray) were investigated. First, Cy-5-labeled mRNAs that were immunoprecipitated with IMP-1 (IP-mRNA) and Cy-3-labeled total RNAs isolated from A549 cells were co-hybridized on cDNA microarrays. Then, to identify the up-regulated genes in A549 cells compared with normal lung tissues, Cy-5-labeled total RNAs isolated from A549 cells and Cy-3-labeled polyA RNAs derived from normal lung (Clontech) ereco-hybridized. Among 27,648 genes screened, a total of 20 transcripts that were both enriched in IMP-1-IP-mRNA(s) (>2-fold intensity) and overexpressed (>2-fold intensity) in A549 cell line compared with normal lung were identified (Table 3). The 20 genes represented a variety of functions including genes involved in signal transduction (SMAD3, RAN), cell adhesion and cytoskeleton (AMIGO2,LASP1), ubiquitination (UBE2S, RNF20), and some phosphatases (PTP4A1, SYNJ2) (Kurisaki A, et al. Mol Cell Biol, 26: 1318-32, 2006.; Rabenau K E, et al. Oncogene, 23:5056-67, 2004.; Strehl S, et al. Oncogene, 22:157-60, 2003.; Liu Z, et al. J Biol Chem, 267: 15829-35, 1992.; Zhu B, et al. Mol Cell, 20: 601-11, 2005.; Stephens B J, et al. Mol Cancer Ther, 4:1653-61, 2005.; Chuang Y, et al. Cancer Res, 64: 8271-5, 2004.). Several of them have been indicated to have important roles in carcinogenesis; for example, involvement of AMIGO2 LASP1, SYNJ2, and PTP4A1 in cell invasion and migration (Rabenau K E, et al. Oncogene, 23:5056-67, 2004.; Strehl S, et al. Oncogene, 22:157-60, 2003.; Stephens B J, et al. Mol Cancer Ther, 4:1653-61, 2005.; Chuang Y, et al. Cancer Res, 64: 8271-5, 2004.).

TABLE 3
List of 22 Candidate mRNAs Associated with the IMP-1 Identified using RNA-
immunoprecipitation and cDNA Microarray

| | | | | | Ratio IMP- | Ratio total |
|---|---|---|---|---|---|---|
| | | | GENE | | 1/total | RNA/normal |
| RANK * | ACCESSION | Hs.ID | NAME | TITLE | RNA | lung |
| 1 | U68019 | 549051 | SMAD3 | SMAD, mothers | 720.08 | 2.66 |
| | | | | against DPP homolog | | |
| | | | | 3 (Drosophila) | | |
| 2 | CA427461 | 10842 | RAN | RAN, member RAS | 81.86 | 3.21 |
| | | | | oncogene family | | |
| 3 | NM_004939 | 440599 | DDX1 | DEAD (Asp-Glu-Ala- | 34.19 | 2.66 |
| | | | | Asp) box polypeptide 1 | | |
| 4 | NM_004209 | 435277 | SYNGR3 | Synaptogyrin 3 | 12.32 | 12.93 |
| 5 | U05569 | 184085 | CRYAA | Crystallin, alpha A | 9.77 | 2.17 |
| 6 | BC050284 | 11747 | YTHDF1 | YTH domain family, | 7.99 | 2.20 |
| | | | | member 1 | | |
| 7 | M91670 | 396393 | UBE2S | Ubiquitin-conjugating | 6.78 | 6.42 |
| | | | | enzyme E2S | | |
| 8 | AY454159 | 121520 | AMIGO2 | Adhesion molecule | 5.65 | 18.09 |
| | | | | with Ig-like domain 2 | | |
| 9 | AA112466 | 554875 | PDF | Peptide deformylase- | 4.18 | 3.93 |
| | | | | like protein | | |
| 10 | AI076810 | 133977 | MGC27277 | Chromosome 1 open | 3.43 | 136.99 |
| | | | | reading frame 67 | | |
| 11 | AI242497 | 151675 | C20orf142 | Chromosome 20 open | 3.21 | 3.79 |
| | | | | reading frame 142 | | |
| 12 | NM_032438 | 486466 | L3MBTL3 | L(3)mbt-like 3 | 3.19 | 3.12 |
| | | | | (Drosophila) | | |
| 13 | D86960 | 497674 | LPGAT1 | Lysophosphatidylglycerol | 3.14 | 7.84 |
| | | | | acyltransferase 1 | | |
| 14 | NM_003463 | 227777 | PTP4A1 | Protein tyrosine | 3.00 | 2.02 |
| | | | | phosphatase type IVA, | | |
| | | | | member 1 | | |
| 15 | BC007560 | 334851 | LASP1 | LIM and SH3 protein 1 | 2.66 | 2.86 |
| 16 | NM_024052 | 187422 | C17orf39 | Chromosome 17 open | 2.57 | 2.77 |
| | | | | reading frame 39 | | |
| 17 | AA191573 | 434494 | SYNJ2 | Synaptojanin 2 | 2.56 | 4.33 |
| 18 | X04217 | 82609 | HMBS | Hydroxymethylbilane | 2.48 | 2.15 |
| | | | | synthase | | |
| 19 | NM_007007 | 369606 | CPSF6 | Cleavage and | 2.24 | 3.02 |
| | | | | polyadenylation | | |
| | | | | specific factor 6, | | |
| | | | | 68 kDa | | |
| 20 | NM_019592 | 168095 | RNF20 | Ring finger protein 20 | 2.01 | 2.52 |

### III. Discussion on the Results

β-actin (ACTB) mRNA is transported to the leading lamellae of chicken-embryo fibroblasts (CEFs) and to the growth cones of developing neurons (Lawrence, J. B. and Singer, R. H. Cell, 45: 407-15, 1986.; Bassell, G. J., et al. J Neurosci, 18: 251-65, 1998.). The localization of ACTB mRNA depends on the “zipcode”, a cis-acting element in the 3′ UTR of the mRNA (Kislauskis, E. H., et al. J Cell Biol, 123: 165-72, 1993.). The respective trans-acting factor, zipcode-binding protein 1 (ZBP1), was identified by affinity purification with the zipcode of ACTB mRNA and it appears to shuttle this RNA to the leading edge of migrating cells (Ross, A. F., et al. Mol Cell Biol, 17: 2158-65, 1997.); homologues of ZBP1 have since been identified in a wide range of organisms including frog, fly, mouse, and human (Mueller-Pillasch, F., et al. Oncogene, 14: 2729-33, 1997.; Deshler, J. O., et al. Science, 276: 1128-31, 1997.; Doyle, G. A., et al. Nucleic Acids Res, 26: 5036-44, 1998.). ZBP1-like proteins contain two RRMs in the N-terminal region and four hnRNP KH (ribonucleoprotein K-homology) domains at the C-terminal end. IMP-1, one of the IGF2 mRNA-binding proteins, is considered to be a member of the ZBP1 family. It exhibits multiple attachments to IGF2 leader-3 mRNA and is over-expressed in several human cancers (Ross, J., et al. Oncogene, 20: 6544-50, 2001.; Ioannidis, P., et al. Int J Cancer, 104: 54-9, 2003.; Ioannidis, P., et al. Cancer Lett, 209: 245-250, 2004.; Gu, L., et al. Int J Oncol, 24: 671-8, 2004.).

In this study, it was confirmed by siRNA experiments that IMP-1 could play a significant role in the tumor cell growth and/or survival. Furthermore, using RNA-immunoprecipitation experiments coupled with cDNA microarrays (IP-microarray), dozens of candidate mRNAs that were likely to be associated with IMP-1 in NSCLC cells were identified (see Table 3). The list included many genes encoding proteins functioning in signal transduction, cell adhesion and cytoskeleton, and those having various types of enzymatic activities. For example, RAN (ras-related nuclear protein) is a small GTP binding protein belonging to the RAS superfamily that is essential for the translocation of RNA and proteins through the nuclear pore complex (Yokoyama, N., et al. Nature, 376: 184-8, 1995.). Ran system is deregulated in certain cellular contexts: this may represent a favoring condition for the onset and propagation of mitotic errors that can predispose cells to become genetically unstable and facilitate neoplastic growth (Di Fiore, B., et al. Cell Cycle, 3: 305-13, 2004.).

Intracellular mRNA transport by RNA-binding proteins has been reported in oocytes and developing embryos of fly and frog, and in somatic cells such as fibroblasts and neurons (King, M. L., et al. Bioessays, 21: 546-57, 1999.; Mowry, K. L. and Cote, C. A. et al. Faseb J, 13: 435-45, 1999.; Lasko, P. et al. J Cell Biol, 150: F51-6, 2000.; Steward, O. Neuron, 18: 9-12, 1997.). IMP-1, which is expressed only in cancers as well as limited normal tissues such as placenta, testis, and fetal tissues, may be required for the transport of certain mRNAs that play essential roles in embryogenesis and carcinogenesis. Therefore, it is postulated that proliferating germ-cells or cancer-cells may actively distribute indispensable mRNAs in cells through the transporting system involved in the IMP-1 protein-mRNA complex. The evidence that IMP-1 associates with various mRNAs encoding proteins involved in cell-cycle progression, cell invasion and migration, and various types of enzymatic activities, supports this premise. In fact, IMP-1 positivity was correlated with tumor extension factor (pT-classification) by clinicopathological investigation using tissue microarray. Further investigations of IMP-1-associated mRNAs may lead to a better understanding of the development of NSCLCs.

Herein, it was further demonstrated that IMP-1 is expressed significantly higher in lung cancer cells than normal lung cells, and that IMP-1 might play an important role in the development/progression of lung cancers. In particular, the results of the instant invention demonstrate that IMP-1 over-expression is associated with lung cancer progression, which, in turn, results in a poor prognosis for patients with lung cancer. Thus, IMP-1 over-expression in resected specimens may be a useful index for application of adjuvant therapy to the patients who are likely to have poor prognosis. Furthermore, our data indicated that up-regulation of IMP-1 is related to growth or survival of cancer cells. Although the molecular mechanisms underlying increased IMP-1 expression levels in many cancer cells have not been elucidated, IMP-1 may represent a promising molecular target for human cancer treatment.

### INDUSTRIAL APPLICABILITY

The gene-expression analysis of cancers described herein, using the combination of laser-capture dissection and genome-wide cDNA microarray, has identified specific genes as targets for cancer prevention and therapy. Based on the expression of a subset of these differentially expressed genes, the present invention provides molecular diagnostic markers for identifying and detecting cancers.

The methods described herein are also useful for the identification of additional molecular targets for prevention, diagnosis, and treatment of cancers. The data provided herein add to a comprehensive understanding of cancers, facilitate development of novel diagnostic strategies, and provide clues for identification of molecular targets for therapeutic drugs and preventative agents. Such information contributes to a more profound understanding of tumorigenesis, and provide indicators for developing novel strategies for diagnosis, treatment, and ultimately prevention of cancers.

All patents, patent applications, and publications cited herein are incorporated by reference in their entirety.

Furthermore, while the invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention. Thus, the invention is intended to be defined not by the above description, but by the following claims and their equivalents.

1. A method for diagnosing lung cancer or a predisposition for developing lung cancer in a subject, comprising the step of determining the expression level of the IMP-1 gene in a subject-derived biological sample, wherein an increase in said expression level as compared to a normal control level of said gene indicates that said subject suffers from or is at a risk of developing lung cancer.

2. The method of claim 1, wherein said IMP-1 expression level is at least 10% greater than the normal control level.

3. The method of claim 1, wherein said expression level is determined by any of the methods selected from the group consisting of:
(a) detecting mRNA of the IMP-1 gene;
(b) detecting a protein encoded by the IMP-1 gene; and
(c) detecting a biological activity of the protein encoded by the IMP-1 gene.

4. The method of claim 1, wherein said subject-derived biological sample comprises an epithelial cell.

5. The method of claim 1, wherein said subject-derived biological sample comprises a cancer cell.

6. The method of claim 1, wherein said subject-derived biological sample comprises a cancerous epithelial cell.

7. The method of claim 1, wherein said lung cancer is non-small cell lung cancer (NSCLC).

8. A method of identifying an agent for treating or preventing lung cancer, which comprises the steps of:
a) contacting a test agent with an IMP-1 polypeptide or a functional fragment thereof;
b) detecting the binding between the IMP-1 polypeptide or functional fragment and the test agent; and
c) selecting the test agent that binds to the polypeptide or fragment.

9. A method of identifying an agent for treating or preventing lung cancer, which comprises the steps of:
a) contacting a test agent with an IMP-1 polypeptide or a functional fragment thereof;
b) detecting the biological activity of the IMP-1 polypeptide or functional fragment; and
c) selecting the test agent that suppresses the biological activity of the polypeptide or fragment as compared to that detected in the absence of the test agent.

10. A method of identifying an agent for treating or preventing lung cancer, which comprises the steps of:
a) contacting a test agent with a cell expressing the IMP-1 gene;
b) detecting the expression level of the IMP-1 gene; and
c) selecting the test agent that reduces the expression level of said gene as compared to that detected in the absence of the test agent.

11. The method of claim 10, wherein said cell is derived from NSCLCs.

12. A method of identifying an agent for treating or preventing lung cancer, which comprises the steps of:
a) contacting a test agent with a cell introduced with a vector that comprises a transcriptional regulatory region of the IMP-1 gene and a reporter gene expressed under the control of said transcriptional regulatory region;
b) measuring the expression or activity of said reporter gene; and
c) selecting the test agent that reduces the expression or activity of said reporter gene as compared to that detected in the absence of the test agent.

13. A method of identifying an agent for treating or preventing lung cancer, which comprises the steps of:
a) contacting a test agent with a cell expressing the IMP-1 protein or functional equivalent thereof and mRNA(s) of one or more gene(s) selected from Table.3;
b) detecting the binding of the IMP-1 protein and the mRNA(s); and
c) selecting the test agent that reduces the binding of the IMP-1 protein and the mRNA(s) as compared to that detected in the absence of the test agent.

14. The method of any one of claims 8 to 13, wherein the lung cancer is NSCLC.

15. A therapeutic agent for treating or preventing lung cancer, which comprises as an active ingredient a pharmaceutically effective amount of an agent selected by any of the methods of claims 8 to 13, and a pharmaceutically acceptable carrier.

16. A therapeutic agent for treating or preventing lung cancer, which comprises a pharmaceutically effective amount of an antisense polynucleotide or siRNA against a polynucleotide encoded by the IMP-1 gene.

17. The therapeutic agent of claim 16, wherein said siRNA comprises the sense strand of the IMP-1 gene comprising the nucleotide sequence of SEQ ID NOs: 9 or 10.

18. The therapeutic agent of claim 17, wherein said siRNA has the general formula 5′-[A]-[B]-[A′]-3′,
wherein [A] is a ribonucleotide sequence corresponding to a sequence of SEQ ID NOs: 9 or 10, [B] is a ribonucleotide loop sequence consisting of 3 to 23 nucleotides, and [A′] is a ribonucleotide sequence complementary to [A].

19. A therapeutic agent for treating or preventing lung cancer, which comprises a pharmaceutically effective amount of an antibody or immunologically active fragment thereof that binds to the IMP-1 polypeptide.

20. The therapeutic agent of any one of claims 15 to 19, wherein the lung cancer is NSCLC.

21. A method for treating or preventing lung cancer in a subject, which comprises the step of administering an agent obtained by any of the methods according to claims 8 to 14.

22. A method for treating or preventing lung cancer in a subject, which comprises the step of administering to said subject the therapeutic agent of any one of claims 15 to 19.

23. A method for treating or preventing lung cancer in a subject, which comprises the step of administering to the subject a pharmaceutically effective amount of an antibody or immunologically active fragment thereof, that binds to the IMP-1 polypeptide.

24. The method of any one of claims 21 to 23, wherein the lung cancer is NSCLC.

25. A method for assessing the prognosis of a patient with lung cancer, which method comprises the steps of:
a) detecting the expression level of the IMP-1 gene in a patient-derived biological sample;
b) comparing the detected expression level to a control level; and
c) determining the prognosis of the patient based on the comparison of (b).

26. The method of claim 25, wherein the lung cancer is NSCLC.

27. The method of claim 25, wherein the control level corresponds to a good prognosis control level and an increase of the expression level as compared to the control level is determined as poor prognosis.

28. The method of claim 27, wherein the IMP-1 expression level is at least 10% greater than said control level.

29. The method of claim 25, wherein said method further comprises the step of determining the expression level of other lung cancer-associated genes.

30. The method of claim 25, wherein said expression level is determined by any one method selected from the group consisting of:
a) detecting mRNA of the IMP-1 gene;
b) detecting the IMP-1 protein; and
c) detecting the biological activity of the IMP-1 protein.

31. The method of claim 25, wherein said expression level is determined by detecting hybridization of a probe to a gene transcript of the IMP-1 gene.

32. The method of claim 31, wherein the hybridization step is carried out on a DNA array.

33. The method of claim 25, wherein said expression level is determined by detecting the binding of an antibody against the IMP-1 protein.

34. The method of claim 25, wherein said biological sample comprises sputum or blood.

35. A double-stranded molecule comprising a sense strand and an antisense strand, wherein the sense strand comprises a ribonucleotide sequence corresponding to a target sequence selected from the group consisting of SEQ ID NOs: 9 and 10, and wherein the antisense strand comprises a ribonucleotide sequence which is complementary to said sense strand, wherein said sense strand and said antisense strand hybridize to each other to form said double-stranded molecule, and wherein said double-stranded molecule, when introduced into a cell expressing the IMP-1 gene, inhibits expression of said gene.

36. The double-stranded molecule of claim 35, wherein said target sequence comprises at least about 10 contiguous nucleotides from the nucleotide sequences of SEQ ID NO: 11.

37. The double-stranded molecule of claim 36, wherein said target sequence comprises from about 19 to about 25 contiguous nucleotides from the nucleotide sequences of SEQ ID NO: 11.

38. The double-stranded molecule of claim 37, wherein said double-stranded molecule is a single ribonucleotide transcript comprising the sense strand and the antisense strand linked via a single-stranded ribonucleotide sequence.

39. The double-stranded molecule of claim 36, wherein the double-stranded molecule is an oligonucleotide of less than about 100 nucleotides in length.

40. The double-stranded molecule of claim 39, wherein the double-stranded molecule is an oligonucleotide of less than about 75 nucleotides in length.

41. The double-stranded molecule of claim 40, wherein the double-stranded molecule is an oligonucleotide of less than about 50 nucleotides in length.

42. The double-stranded molecule of claim 41, wherein the double-stranded molecule is an oligonucleotide of less than about 25 nucleotides in length.

43. The double-stranded molecule of claim 42, wherein the double stranded molecule is an oligonucleotide of between about 19 and about 25 nucleotides in length.

44. A vector encoding the double-stranded molecule of claim 35.

45. The vector of claim 44, wherein the vector encodes a transcript having a secondary structure and comprises the sense strand and the antisense strand.

46. The vector of claim 44, wherein the transcript further comprises a single-stranded ribonucleotide sequence linking said sense strand and said antisense strand.

47. A vector comprising a polynucleotide comprising a combination of a sense strand nucleic acid and an antisense strand nucleic acid, wherein said sense strand nucleic acid comprises nucleotide sequence of SEQ ID NOs: 9 and 10, and said antisense strand nucleic acid consists of a sequence complementary to the sense strand.

48. The vector of claim 47, wherein said polynucleotide has the general formula 5′-[A]-[B]-[A′]-3′
wherein [A] is a nucleotide sequence of SEQ ID NOs: 9 and 10; [B] is a nucleotide sequence consisting of 3 to 23 nucleotides; and [A′] is a nucleotide sequence complementary to [A].

49. An antibody recognizing IMP-1 but not recognizing IMP-2 and IMP-3.

50. The antibody of claim 49, which binds the antigen comprising peptide selected from the group consisting of SEQ ID NO: 5 or SEQ ID NO: 6.
